(19) United States
(12) Patent Application Publication
(10) Pub. No.: US 2024/0376194 A1
(43) Pub. Date: Nov. 14, 2024

(54) D3-BINDING MOLECULES AND USES THEREOF

(71) Applicant: WUXI BIOLOGICS IRELAND LIMITED, Dundalk, Co Louth (IE)

(72) Inventors: Yunying CHEN, Shanghai (CN); Yongqing CHENG, Shanghai (CN); Xia WANG, Shanghai (CN); Jijie GU, Shanghai (CN)

(73) Assignee: WUXI BIOLOGICS IRELAND LIMITED, Dundalk, Co Louth (IE)

(21) Appl. No.: 18/692,311

(22) PCT Filed: Sep. 16, 2022
(86) PCT No.: PCT/CN2022/119334

§ 371 Date: Mar. 14, 2024

(30) Foreign Application Priority Data
Sep. 17, 2021 (WO) PCT/CN2021/119011

(51) Int. Cl. C07K 16/28

(52) U.S. Cl. CPC C07K 16/28; C07K 2317/22; C07K 2317/24; C07K 2317/33; C07K 2317/569; C07K 2317/76; C07K 2317/77; C07K 2317/92; C07K 2317/94

(57) ABSTRACT

Provided D3-binding molecules, including anti-D3antibodies, and their uses.

### CROSS-REFERENCE TO RELATED APPLICATIONS

This application claims benefit of priority of International Patent Application No. PCT/CN2021/119011 filed on Sep. 17, 2021, the disclosure of which is incorporated by reference herein in its entirety.

### SEQUENCE LISTING

This application incorporates by reference a Sequence Listing submitted with this application.

### FIELD

This application generally relates to Delta Like Canonical Notch Ligand 3 (D3)-binding molecules, including anti-D3 antibodies, and their uses.

### BACKGROUND

Delta Like Canonical Notch Ligand 3 (D3 or DLL3) is a type I transmembrane protein that belongs to the DSL family of Notch ligands. It is normally expressed exclusively on intracellular membranes, especially the Golgi apparatus. Additional Notch family ligands include Delta Like Canonical Notch Ligand 1 (D1), Delta Like Canonical Notch Ligand 4 (D4), Jagged Canonical Notch Ligand 1 (J1), and Jagged Canonical Notch Ligand 2 (J2). Except for D3, the other ligands can activate Notch signaling. D3 acts as an inhibitor of Notch signaling by interfering with the binding between Notch and its ligands. D3 is highly expressed on lung tumor cell surface, including small cell lung cancer (SCLC) and large cell neuroendocrine carcinoma (LCNEC). While it is normally expressed exclusively on the intracellular membranes, D3 is a potential therapeutic tumor target for any tumors that express D3, including SCLC and LCNEC.

Lung cancer is the most common cause of cancer death and about 2 million people are diagnosed with lung cancer every year in the world; about 15% of all lung cancer cases are SCLC, which is the most aggressive form of lung cancer with very limited therapeutic options: surgery, chemotherapy and radiation therapy. In 2019, FDA approved Atezolizumab (anti-PD-L 1) for first-line treatment of SCLC, while the limited 2-month benefit highlights the need for development of additional therapies.

There is still a need to find new D3-targeted agents, including D3-binding molecules such as monoclonal antibodies for uses, including as antibody conjugates such as ADCs or as bispecific antibodies and for CAR-T therapy development.

### SUMMARY

The present disclosure is directed to compounds, methods, compositions and articles of manufacture that provide D3-binding molecules with improved efficacy. The benefits provided by the present disclosure are broadly applicable in the field of antibody therapeutics and diagnostics and may be used in conjunction with other therapeutics such as antibodies that react with a variety of targets.

The present disclosure provides D3-binding molecules, such as monoclonal antibodies, that can specifically bind to human D3 and are cross-reactive with cynomolgus monkey and/or mouse D3. Such D3-binding molecules provide certain advantages compared to the agents, compositions and/or methods currently used and/or known in the art. These advantages include internalization potency, improved therapeutic and pharmacological properties, increased specificity, improved safety profile, reduced immunogenicity, and other advantageous properties such as improved ease of preparation or reduced costs of goods, higher stability especially as compared to candidate drugs already known in the art.

In the present disclosure, D3-binding molecules, such as monoclonal antibodies, against D3 which can be used to treat D3-overexpressing tumor have been developed.

The present disclosure provides D3-binding molecules, nucleic acid molecules encoding the same, expression vectors and host cells used for the expression of D3-binding molecules, and methods for using D3-binding molecules. D3-binding molecules of the present disclosure provide potent agents for the treatment of multiple cancers (including lung cancer) via modulating human immune function.

In some embodiments, the present disclosure provides a D3-binding molecule comprising at least one immunoglobulin single variable domain (e.g. VHH domain) that specifically binds to D3, such as human D3, cyno D3 and/or mouse DLL-3. In some embodiments, the single variable domain comprises CDR1, CDR2 and CDR3, and wherein:
the CDR1 comprises an amino acid sequence as set forth in SEQ ID NO: 1, 4, 7 or 10;
the CDR2 comprises an amino acid sequence as set forth in SEQ ID NO: 2, 5, 8 or 11; and
the CDR3 comprises an amino acid sequence as set forth in SEQ ID NO: 3, 6 or 9.

In some embodiments, the single variable domain as disclosed herein comprises:
(A) a CDR1 as set forth in SEQ ID NO: 1; a CDR2 as set forth in SEQ ID NO: 2; and a CDR3 as set forth in SEQ ID NO: 3;
(B) a CDR1 as set forth in SEQ ID NO: 4; a CDR2 as set forth in SEQ ID NO: 5; and a CDR3 as set forth in SEQ ID NO: 6;
(C) a CDR1 as set forth in SEQ ID NO: 7; a CDR2 as set forth in SEQ ID NO: 8; and a CDR3 as set forth in SEQ ID NO: 9; or
(D) a CDR1 as set forth in SEQ ID NO: 10; a CDR2 as set forth in SEQ ID NO: 11; and a CDR3 as set forth in SEQ ID NO: 6.

In some embodiments, the single variable domain as disclosed herein comprises:
(A) a CDR1 as set forth in SEQ ID NO: 27; a CDR2 as set forth in SEQ ID NO: 28 or 56; and a CDR3 as set forth in SEQ ID NO: 29;
(B) a CDR1 as set forth in SEQ ID NO: 38; a CDR2 as set forth in SEQ ID NO: 39; and a CDR3 as set forth in SEQ ID NO: 40; or
(C) a CDR1 as set forth in SEQ ID NO: 49; a CDR2 as set forth in SEQ ID NO: 50; and a CDR3 as set forth in SEQ ID NO: 51;
wherein the CDR numbering is according to Contact numbering system.

In some embodiments, the single variable domain as disclosed herein comprises:
(A) an amino acid sequence as set forth in any one of SEQ ID NOs: 12-18 and 55;
(B) an amino acid sequence at least 85%, 90%, or 95% identical to the amino acid sequence as set forth in any one of SEQ ID NOs: 12-18 and 55 yet the specific binding affinity to D3 is maintained (e.g., substantially maintained, for example, at least 50%, at least 60%, at least 70%, at least 80%, at least 90%, at least 95%); or
(C) an amino acid sequence with addition, deletion and/or substitution of one or more (e.g. 1, 2 or 3) amino acids compared with the amino acid sequence as set forth in any one of SEQ ID NOs: 12-18 and 55 yet the specific binding affinity to D3 is maintained (e.g., substantially maintained, for example, at least 50%, at least 60%, at least 70%, at least 80%, at least 90%, at least 95%).

In some embodiments, D3-binding molecules as disclosed herein comprise one or more substitutions, additions, and/or deletions of amino acids in the framework regions, e.g. FRW1, FRW2, FRW3, and/or FRW4 of a single variable domain (e.g., VHH). In some embodiments, FRW1 at the N terminal and/or FRW4 at the C terminal of the single variable domain is truncated, e.g. truncated by no more than 5, 4, 3, 2, or 1 amino acid(s).

In some embodiments, a single variable domain (e.g., VHH) comprises an amino acid sequence as set forth in any one of SEQ ID NOs: 12-18 and 55.

In some embodiments, a D3-binding molecule as disclosed herein further comprises one or more human IgG constant domains, such as one or more human IgG1, IgG2, IgG3 or IgG4 constant domains. In some embodiments, the IgG constant domain is a human IgG1 constant domain or a variant thereof. An example of an amino acid sequence of IgG1 constant domains is as set forth in SEQ ID NO: 19. In some embodiments, a D3-binding molecule comprises a variant of one or more human IgG1 constant domains, e.g. an IgG1 Fc with L234A/L235A substitutions, according to EU numbering.

In some embodiments, a D3-binding molecule as disclosed herein has one or more of the following properties:
(a) binds to human D3, cyno D3 and/or mouse D3 with EC50s at nM grade, as measured by ELISA or FACS;
(b) shows dose-dependent internalization potency in human D3 expressing cells; and
(c) binds to human D3 with a KD of no more than 0.1 nM, as measured by SPR.

In some embodiments, a D3-binding molecule as disclosed herein is a chimeric antibody, a humanized antibody or a fully human antibody. In some embodiments, the D3-binding molecule is a dimer.

In some embodiments, a D3-binding molecule as disclosed herein comprises a single variable domain as set forth in any one of SEQ ID NOs: 12-18 and 55, and IgG constant domains as set forth in SEQ ID NO: 19.

In some embodiments, the present disclosure provides a nucleic acid molecule comprising a nucleic acid sequence encoding a D3-binding molecule as disclosed herein, such as a D3-binding molecule comprising a single variable domain (e.g., VHH).

In some embodiments, the present disclosure provides a vector comprising a nucleic acid molecule as disclosed herein.

In some embodiments, the present disclosure provides a host cell comprising an expression vector or a nucleic acid molecule as disclosed herein.

In some embodiments, the present disclosure provides a pharmaceutical composition comprising a D3-binding molecule as disclosed herein and a pharmaceutically acceptable carrier.

In some embodiments, the present disclosure provides a method for preparing a D3-binding molecule which comprises expressing the D3-binding molecule in a host cell as disclosed herein and isolating the D3-binding molecule from the host cell.

In some embodiments, the present disclosure provides a method of modulating a D3-related immune response in a subject, comprising administering a D3-binding molecule as disclosed herein to the subject such that the D3-related immune response in the subject is modulated.

In some embodiments, the present disclosure provides a method for treating or preventing a D3 positive or D3 overexpressed cancer in a subject comprising administering an effective amount of a D3-binding molecule or a pharmaceutical composition as disclosed herein to the subject. In some embodiments, the cancer is lung cancer, including, for example, SCLC and LCNEC.

In some embodiments, the present disclosure provides use of a D3-binding molecule as disclosed herein in the manufacture of a medicament for diagnosing, treating or preventing a D3 positive cancer.

In some embodiments, the present disclosure provides a D3-binding molecule as disclosed herein for use in diagnosing, treating or preventing a D3 positive cancer.

In some aspects, the present disclosure is directed to kits or devices and associated methods that employ a D3-binding molecule as disclosed herein, or pharmaceutical compositions as disclosed herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the binding molecules, methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1 shows exemplary binding results of antibodies with immobilized WT115-hPro1.ECD.his measured by ELISA.

FIGS. 2a and 2b show exemplary binding results of antibodies with WT115-293F.hPro1.2E5 cells measured by FACS.

FIGS. 3a and 3b show exemplary binding results of antibodies with WT115-Flpin293.cPro1.pool cells, measured by FACS.

FIGS. 4a and 4b show exemplary binding results of antibodies on immobilized WT115-MBP-mPro1.ECD.hFc, measured by ELISA.

FIGS. 5a and 5b show exemplary internalization results of antibodies by WT115-293F.hPro1.2E5 cells.

FIGS. 6a-6d show exemplary epitope binning results of antibodies on immobilized WT115-hPro1.ECD.his by ELISA. FIG. 6a, binning with WT1156-P3R2-1C2-uIgG1; FIG. 6b, binning with WT1156-P3R2-1H6-uIgG1; FIG. 6c, binning with WT1156-P8R2-1H1-uIgG1; FIG. 6d, binning with WT115-BMK1.

FIG. 7a shows an exemplary ELISA binding result of antibodies on soluble WT115-hPro1.ECD.his or truncated proteins by ELISA. FIG. 7b shows ELISA binding of antibodies on immobilized WT115-hPro1.ECD.his or truncated proteins by ELISA. FIG. 7c shows the diagrams of the truncated protein.

FIG. 8 shows exemplary cross-family binding results of antibodies with human D1 and human D4, measured by ELISA.

FIG. 9 shows exemplary serum stability test results of WT1156-P3R2-1C2-z109-uIgG1.

FIG. 10 shows an alignment of exemplary immunoglobulin single variable domains WT1156-P3R2-1C2 (1C2), WT1156-P3R2-1C2-z102 (1C2-z102), WT1156-P3R2-1C2-z109 (1C2-z109), WT1156-P3R2-1C9 (1C9), WT1156-P3R2-1H6 (1H6), WT1156-P3R2-1H6-z100 (1H6-z100), and WT1156-P8R2-1H1 (1H1). Boundaries of CDRs are indicated by Kabat, AbM, Chothia, Contact, and IMGT numbering.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised”, is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are well known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are well known and commonly used in the art.

### Definitions

In order to better understand the disclosure, the definitions and explanations of the relevant terms are provided as follows.

The term “antibody” (e.g. anti-D3 antibody) and “antigen-binding molecule” (e.g. D3-binding molecule) are used interchangeably in the broadest sense and encompass any form of antibody that exhibits the desired biological or binding activity. It covers, but is not limited to, humanized antibodies, fully human antibodies, chimeric antibodies and single-domain antibodies (sdAbs, comprising just one chain, which is typically similar to a heavy chain), as well as fragments of any of the foregoing as long as they exhibit the desired antigen-binding activity, including, for example, an antibody comprising at least one VHH domain. A conventional antibody comprises a heavy chain(s) and a light chain(s). Heavy chains may be classified into μ, δ, γ, α and εE, which define isotypes of an antibody as IgM, IgD, IgG, IgA and IgE, respectively. A heavy chain can comprise a heavy chain variable region (V_{H}) and a heavy chain constant region (C_{H}). A heavy chain can comprise one or more constant regions, for example, 3 constant regions (C_{H}1, C_{H}2 and C_{H}3). A light chain can comprise a light chain variable region (V_{L}) and a light chain constant region (C_{L}). A V_{H} and a V_{L} region can further be divided into hypervariable regions (called complementary determining regions (CDRs)), which are interspaced by relatively conservative regions (called framework regions (FRW)). A V_{H} and a V_{L} can comprise 3 CDRs (Complementarity determining regions) and 4 FRs (Framework regions) in the following order: FRW1, CDR1, FRW2, CDR2, FRW3, CDR3, FRW4 from N-terminal to C-terminal. Antibodies can be of different antibody isotypes, for example, IgG (e.g., IgG1, IgG2, IgG3 or IgG4 subtype), IgA1, IgA2, IgD, IgE or IgM antibody.

The term “Fc region” is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding region or binding domain (e.g., an antibody variable region or domain, including a VHH domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, (e.g., substituting, addition, or deletion) preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. A variant Fc region can possess at least about 80% homology with a native sequence Fc region and/or with an Fc region of a parent polypeptide, or at least about 90% homology therewith, for example, at least about 95% homology therewith. The variant Fc region herein described herein may have a loss of effector function (e.g., silent Fc).

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

The term “immunoglobulin single variable domain” or “single variable domain” or “VHH domain” or “VHH” or “heavy chain only antibody variable domain” may be used interchangeably herein and refers to a single chain antigen binding domain that is capable of binding to an antigen or epitope, independently of a different variable domain. A VHH domain (e.g. variable domain of a heavy chain antibody) represents the smallest known antigen-binding unit generated by adaptive immune responses (Koch-Nolte F. et al., FASEB J. November; 21(13):3490-8. Epub 2007 Jun. 15 (2007)). A VHH domain may be a human domain, but also includes a single domain from other species such as rodent, nurse shark and Camelid VHH domains. Camelid VHH are immunoglobulin single variable domain polypeptides that are derived from species including camel, llama, alpaca, dromedary, and guanaco, which produce heavy chain antibodies naturally devoid of light chains. Such VHH domains may be humanized according to standard techniques available in the art and are considered as “single domain antibodies”. As used herein, VHH includes camelid VHH domains and humanized VHH domains.

The term “humanized antibody” is intended to refer to antibodies in which CDR sequences derived from the germline of another mammalian species, such as a mouse, llama or alpaca, have been grafted onto human framework sequences. Additional framework region modifications may be made within the human framework sequences.

The term “Ka”, as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “KD” as used herein, is intended to refer to the dissociation constant of a particular antibody-antigen interaction, which is obtained from the ratio of Kd to Ka (e.g., Kd/Ka) and is expressed as a molar concentration (M). A preferred method for determining the Kd of an antibody is by using surface plasmon resonance, preferably using a biosensor system such as a Biacore® system.

The term “specific binding” or “specifically binds” as used herein refers to a non-random binding reaction between two molecules, such as for example between an antibody and an antigen.

The term “high affinity”, as used herein, refers to a D3 binding molecule such as an antibody having a KD of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen.

The term “EC₅₀”, as used herein, which is also termed as “half maximal effective concentration” refers to the concentration of a drug, antibody or toxicant which induces a response halfway between the baseline and maximum after a specified exposure time. In the context of the present disclosure, EC₅₀ is expressed in the unit of “nM”.

The term “epitope”, as used herein, refers to a portion of an antigen that an immunoglobulin or antibody specifically binds to. “Epitope” is also known as “antigenic determinant”. Epitope or antigenic determinant generally comprises chemically active surface groups of a molecule such as amino acids, carbohydrates or sugar side chains, and generally has a specific three-dimensional structure and a specific charge characteristic. For example, an epitope generally comprises at least 3, 4, 5, 6, 7, 8, 9, 10, 11, 12, 13, 14 or 15 consecutive or non-consecutive amino acids in a unique steric conformation, which may be “linear” or “conformational”. See, for example, Epitope Mapping Protocols in Methods in Molecular Biology, Vol. 66, G. E. Morris, Ed. (1996). In a linear epitope, all the interaction sites between a protein and an interaction molecule (e.g., an antibody) are present linearly along the primary amino acid sequence of the protein. In a conformational epitope, the interaction sites span over amino acid residues that are separate from each other in a protein. Antibodies may be screened depending on competitiveness of binding to the same epitope by conventional techniques known by a person skilled in the art. For example, studies on competition or cross-competition may be conducted to obtain antibodies that compete or cross-compete with each other for binding to antigens. High-throughput methods for obtaining antibodies binding to the same epitope, which are based on their cross-competition, are described in an international patent application WO 03/48731.

The term “isolated antibody”, as used herein, is intended to refer to an antibody that is substantially free of other antibodies having different antigenic specificities (e.g., an isolated antibody that specifically binds a D3 protein is substantially free of antibodies that specifically bind antigens other than D3 proteins). An isolated antibody that specifically binds a human D3 protein may, however, have cross-reactivity to other antigens, such as D3 proteins from other species. Moreover, an isolated antibody can be substantially free of other cellular material and/or chemicals.

The term “vector”, as used herein, refers to a nucleic acid vehicle which can have a polynucleotide inserted therein. When the vector allows for the expression of the protein encoded by the polynucleotide inserted therein, the vector is called an expression vector. The vector can have carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise an origin of replication.

The term “host cell”, as used herein, refers to a cell into which a vector can be introduced, including, but not limited to, a prokaryotic cell such as E. coli or Bacillus subtilis, a fungal cell such as yeast cell or Aspergillus, an insect cell such as S2 Drosophila cell or Sf9, and an animal cell such as fibroblast, CHO cell, COS cell, NSO cell, HeLa cell, BHK cell, HEK 293 cell or human cell.

The term “identity”, as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (e.g., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity”, as used herein, refers to an ability to stimulate formation of specific antibodies or sensitized lymphocytes in organisms. It not only refers to a property of an antigen to stimulate a specific immunocyte to activate, proliferate and differentiate so as to finally generate immunologic effector substance such as antibody and sensitized lymphocyte, but also refers to a specific immune response that antibody or sensitized T lymphocyte can be formed in an immune system of an organism after stimulating the organism with an antigen. Immunogenicity is an important property of an antigen. Whether an antigen can successfully induce the generation of an immune response in a host depends on several factors, including properties of an antigen, reactivity of a host, and immunization means.

The term “transfection” or “transfect”, as used herein, refers to a process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197.

The term “SPR” or “surface plasmon resonance”, as used herein, refers to and includes an optical phenomenon that allows for an analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example and Jönsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS”, as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09.). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably humans.

The term “condition associated with D3” or “condition related to D3”, as used herein, refers to any condition that is caused by, exacerbated by, or otherwise linked to increased or decreased (generally increased) expression or activities of D3 (e.g. a human D3).

The term “cancer”, as used herein, refers to any tumor or any malignant cell growth or proliferation, primary or metastasis-mediated, including solid tumors and non-solid tumors such as leukemia.

The term “treatment”, “treating” or “treated”, as used herein in the context of treating a condition, pertains generally to treatment or therapy, whether of a human or an animal, in which some desired therapeutic effect is achieved, for example, inhibition of the progress of a condition, and includes a reduction in the rate of progress, a halt in the rate of progress, regression of the condition, amelioration of the condition, and cure of the condition. Treatment as a prophylactic measure (e.g., prophylaxis, prevention) is also included. For cancer, “treating” may refer to a dampening or slowing of a tumor or malignant cell growth, proliferation, or metastasis, or some combination thereof. For tumors, “treatment” includes removal of all or part of a tumor, inhibiting or slowing tumor growth and metastasis, preventing or delaying the development of a tumor, or some combination thereof.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. For example, a “therapeutically-effective amount,” of a D3-binding molecule refers to an amount or concentration effective to treat a human D3-related disease or condition.

The term “host cell”, as used herein, refers to a cell with the introduction of exogenous polynucleotides.

The term “pharmaceutically acceptable”, as used herein, means that the vehicle, diluent, excipient and/or salts thereof, are chemically and/or physically compatible with other ingredients in the formulation, and physiologically compatible with the recipient.

As used herein, the term “a pharmaceutically acceptable carrier and/or excipient” refers to a carrier, stabilizer, and/or excipient pharmacologically and/or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to a pH adjuster, surfactant, adjuvant or an ionic strength enhancer. For example, a pH adjuster includes, but is not limited to, phosphate buffer; a surfactant includes, but is not limited to, cationic, anionic, or non-ionic surfactant, e.g., Tween-80; an ionic strength enhancer includes, but is not limited to, sodium chloride. Carriers, excipients, or stabilizers are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is a exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington's Pharmaceutical Sciences (1990) Mack Publishing Co., Easton, PA. Compositions, including pharmaceutical compounds, may contain a prophylactically or therapeutically effective amount of a D3-binding agent (e.g., an anti-D3 antibody), for example, in isolated or purified form, together with a suitable amount of carrier so as to provide the form for proper administration to the subject (e.g., patient). The formulation should suit the mode of administration.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments. Aluminum hydroxide adjuvant is more commonly used in clinical trials.

### D3-Binding Molecules

In some aspects, the disclosure provides D3-binding molecules. A D3-binding molecule, in a general sense, may include any molecule that specifically binds to D3. In some circumstances, a “D3-binding molecule” may include a “D3 antagonist” and an “anti-D3 antibody”. “D3 antagonist” refers to any chemical compound or biological molecule that blocks D3 activities. “Anti-D3 antibody” includes, but not limited to, a chimeric antibody, a humanized antibody, a human antibody or a single-domain antibody. A D3-binding molecule is not limited to a polypeptide or a protein and may comprise other components such as nucleotides, hybrids, glucans and a combination thereof. As exemplified herein, a D3-binding molecule may be an anti-D3 antibody or anti-D3 fusion protein.

In some embodiments, D3-binding molecules as disclosed herein comprise at least one VHH that specifically binds to D3. Further, a D3-binding molecule may be a single-domain antibody and comprising one VHH. For example, a single-domain antibody is able to bind selectively to a specific antigen (e.g., D3). In some embodiments, a D3-binding molecule comprises a VHH fused to an immunoglobulin Fc region, for example, an Fc region of IgG (e.g., IgG4 or IgG1). In some embodiments, the Fc region is an Fc region of human IgG1. By fusing a VHH to an Fc region, it may be more efficient to recruit effector functions. Also, fusion of a VHH to an Fc region may help a D3-binding molecule to form a dimer and may also help the extension of the half life of the D3-binding molecule in vivo.

As known in the art, VHH molecules derived from Camelidae antibodies are among the smallest intact antigen-binding domains known (approximately 15 kDa, or 10 times smaller than a conventional IgG) and hence are well suited towards delivery to dense tissues and for accessing the limited space between macromolecules.

VHHs as disclosed herein may be made by the skilled artisan according to methods known in the art or any future method. For example, VHHs may be obtained using methods known in the art such as by immunizing a camel and obtaining hybridoma's therefrom, or by cloning a library of VHHs of the disclosure using molecular biology techniques known in the art and subsequent selection by using phage display.

For example, a VHH can be obtained by immunization of llamas or alpacas with the desired antigen and subsequent isolation of the mRNA coding for heavy-chain antibodies. By reverse transcription and polymerase chain reaction, a gene library of single-domain antibodies containing several million clones is produced. Screening techniques like phage display and ribosome display help to identify the clones binding the antigen. One technique is phage display in which a library of (e.g., human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., Barbas et al., Proc. Natl. Acad. Sci. USA 88:7978-7982 (1991)).

When potent clones have been identified, their DNA sequence is optimized, for example, by affinity maturation or humanization. Humanization may prevent immunological reactions of the human organism against the antibody.

Accordingly, the VHHs can be obtained (1) by isolating the VHH domain of a naturally occurring heavy chain antibody; (2) by expression of a nucleotide sequence encoding a naturally occurring VHH domain; (3) by “humanization” (as described below) of a naturally occurring VHH domain or by expression of a nucleic acid encoding a such humanized VHH domain; (4) by “camelization” of a naturally occurring VH domain from any animal species, in particular a species of mammal, such as from a human being, or by expression of a nucleic acid encoding such a camelized VH domain; (5) by “camelisation” of a “domain antibody” or “Dab” as described by Ward et al (supra), or by expression of a nucleic acid encoding such a camelized VH domain; (6) using synthetic or semi-synthetic techniques for preparing proteins, polypeptides or other amino acid sequences; (7) by preparing a nucleic acid encoding a VHH using techniques for nucleic acid synthesis, followed by expression of the nucleic acid thus obtained; (8) subjecting heavy chain antibodies or VHHs to affinity maturation, to mutagenesis (e.g. random mutagenesis or site-directed mutagenesis) and/or any other technique(s) in order to increase the affinity and/or specificity of the VHH; and/or (9) by any combination of the foregoing. Suitable methods and techniques for performing the foregoing will be clear to the skilled person based on the disclosure herein and, for example, include methods and techniques described in more detail herein.

Single-domain antibodies are usually generated by PCR cloning of variable domain repertoire from blood, lymph node, or spleen cDNA obtained from immunized animals into a phage display vector. Antigen-specific single-domain antibodies are commonly selected by panning phase libraries on immobilized antigen, for example, antigen coated onto the plastic surface of a test tube, biotinylated antigens immobilized on Streptavidin beads, or membrane proteins expressed on the surface of cells. The affinity of sdAbs can often been improved by mimicking this strategy in vitro, for example, by site directed mutagenesis of the CDR regions and further rounds of panning on immobilized antigen under conditions of increased stringency (higher temperature, high or low salt concentration, high or low pH, and low antigen concentrations) (Wesolowski et al., Single domain antibodies: promising experimental and therapeutic tools in infection and immunity. Med Microbiol Immunol (2009) 198: 157-174).

Methods for preparing a VHH specifically binding to an antigen or epitope was described in references, for example: R. van der Linden et al., Journal of Immunological Methods, 240(2000) 185-195; Li et al., J Biol Chem., 287(2012)13713-13721; Deffar et al., African Journal of Biotechnology Vol. 8(12), pp. 2645, 17 June, 2009 and WO 94/04678.

In some embodiments, a VHH may be truncated at the N-terminus or C-terminus such that it comprises only a partial FRW1 and/or FRW4, or lacks one or both of those framework regions, so long as the VHH substantially maintains antigen binding and specificity (e.g., substantially maintained, for example, at least 50%, at least 60%, at least 70%, at least 80%, at least 90%, at least 95%).

The present disclosure also provides D3-binding molecules with a masking moiety and/or cleavable moiety in which one or more of the D3-binding domains of the D3-binding molecules are masked (e.g., via a masking moiety) and/or activatable (e.g., via a cleavable moiety). Technologies for masking of a D3-binding molecule (e.g., an antibody) are well known in the art, including SAFE body masking technology (see, e.g., US 2019/0241886) and Probody masking technology (see, e.g., US 2015/0079088). Such technologies can be used to generate a D3-binding molecule (e.g., an antibody) that is masked and/or activatable. Such masked and/or activatable D3-binding molecules (e.g., antibodies) are useful for the preparation of conjugates, including immunoconjugates, antibody-drug conjugates (ADCs), masked ADCs and activatable antibody-drug conjugates (AADCs), comprising any one of the D3-binding molecules (e.g., antibodies) of the present disclosure, including those directly or indirectly linked to another agent such as a drug. For example, D3-binding molecules of the present disclosure may be covalently bound by a synthetic linker to one or more agents such as drugs.

If desired, a D3-binding molecule is linked or conjugated (directly or indirectly) to a moiety with effector function, such as cytotoxic activity (e.g., a chemotherapeutic moiety or a radioisotope) or immune recruitment activity. Moieties that are linked or conjugated (directly or indirectly) include drugs that are cytotoxic (e.g., toxins such as auristatins) or non-cytotoxic (e.g., signal transduction modulators such as kinases or masking moieties that mask one or more binding domains of a D3-binding molecule, or cleavable moieties that allow for activating a D3-binding molecule by cleaving of a cleavable moiety to unmask one or more binding domains of a D3-binding molecule in the tumor microenvironment, in the form of masked conjugates. Moieties that promote immune recruitment can include other antigen-binding agents, such as viral proteins that bind selectively to cells of the innate immune system. Alternatively or in addition, a D3-binding molecule is optionally linked or conjugated (directly or indirectly) to a moiety that facilitates isolation from a mixture (e.g., a tag) or a moiety with reporter activity (e.g., a detection label or reporter protein). It will be appreciated that the features of a D3-binding molecule described herein extend also to a polypeptide comprising a D3-binding molecule fragment.

In some embodiments, D3-binding molecules described herein may be linked or conjugated (directly or indirectly) to a polypeptide, which can result in the generation of an activatable antibody. In some embodiments, a D3-binding molecule is linked or conjugated (directly or indirectly) to an agent. In some embodiments, the agent is a drug, resulting in an ADC or an AADC when the antibody of the ADC comprises a masking moiety and a cleavable moiety.

In some embodiments, D3-binding molecules described herein are conjugated or recombinantly linked (directly or indirectly) to a therapeutic agent (e.g., a cytotoxic agent) or to a diagnostic or detectable agent. The conjugated or recombinantly linked antibodies, including masked or activatable conjugates, can be useful, for example, for treating or preventing a disease, disorder or condition, such as a cancer or a tumor.

Diagnosis and detection can be accomplished, for example, by coupling a D3-binding molecule to detectable substances including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (131I, 1251, 1231, and 1211), carbon (14C), sulfur (35S), tritium (3H), indium (115In, 113In, 1121n, and 11 In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Conjugates of an antibody and agent, including wherein the agent is a drug for the preparation of ADC or an AADC, may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone) benzoate). The present disclosure further contemplates that conjugates of antibodies and agents, including wherein the agent is a drug for the preparation of an ADC or AADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of ADC or AADC, have been based on random conjugation chemistries involving the F-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

D3-binding molecules described herein may be monospecific, bispecific, trispecific or of greater multispecificity. Such agents may include antibodies. Multispecific antibodies, such as bispecific antibodies, are monoclonal antibodies that have binding specificities for at least two different targets (e.g., antigens) or two different epitopes on the same target (e.g., a bispecific antibody directed to D3 with a first binding domain for a first epitope of D3, and a second binding domain for a second epitope of D3. In some embodiments, the multispecific (e.g., bispecific) antibodies can be constructed based on the sequences of the antibodies described herein. In some embodiments, the multispecific antibodies described herein are bispecific antibodies. In some embodiments, bispecific antibodies are mouse, chimeric, human or humanized antibodies. In some embodiments, one of the binding specificities of the multispecific antibody is for D3 and the other is for any other target (e.g., antigen). In some embodiments, a multispecific (e.g., bispecific) antibody can comprise more than one target (e.g., antigen) binding domain, in which different binding domains are specific for different targets (e.g., a first binding domain that binds to D3 and a second binding domain that binds another target (e.g., antigen), such as an immune checkpoint regulator (e.g., a negative checkpoint regulator). In some embodiments, multispecific (e.g., bispecific) antibody molecules can bind more than one (e.g., two or more) epitopes on the same target (e.g., antigen). In some embodiments, one of the binding specificities is D3 and the other is for one or more of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092.

Methods for making multispecific antibodies are known in the art, for example, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The present disclosure provides humanized antibodies that bind D3. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind D3 may be produced using techniques known to those skilled in the art (e.g., Zhang et al., Molecular Immunology, 42(12): 1445-1451, 2005; Hwang et al., Methods, 36(1): 35-42, 2005; Dall'Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567).

A D3-binding molecule may be described as an anti-D3 antibody in the following sections.

Anti-D3 Antibodies with Functional Properties

Antibodies of the disclosure including, for example, antibodies comprising at least one VHH domain, are characterized by particular functional features or properties of the antibodies. In some embodiments, the antibodies have one or more of the following properties:
(a) bind to human D3, cyno D3 and mouse D3 with EC50 at nM grade, as measured by ELISA or FACS;
(b) show dose-dependent internalization potency in human cells engineered to express D3; and
(c) bind to human D3 ECD with a KD no more than 0.1 nM, as measured by SPR.

An antibody of the disclosure binds to cell surface D3 with high affinity. The binding of an antibody of the disclosure to D3 can be assessed using one or more techniques well established in the art, for example, ELISA. The binding specificity of an antibody of the disclosure can also be determined by monitoring binding of the antibody to cells expressing a D3 protein, e.g., by flow cytometry. For example, an antibody can be tested by a flow cytometry assay (e.g., FACS) in which the antibody is reacted with a cell line that expresses human D3, such as CHO cells and 293 cells that have been transfected to express D3 on their cell surface. Additionally or alternatively, the binding of the antibody, including the binding kinetics (e.g., Kd value) can be tested in BIAcore binding assays. Still other suitable binding assays include ELISA assays, for example using a recombinant D3 protein. For example, an antibody of the disclosure binds to a cell surface D3 (e.g., human D3 ECD) protein with a KD of 1×10⁻⁷ M or less, 5×10⁻⁸ M or less, 2×10⁻⁸ M or less, 5×10⁻⁹ M or less, 4×10⁻⁹ M or less, 3×10⁻⁹ M or less, 2×10⁻⁹ M or less, 1×10⁻⁹ M or less, 5×10⁻¹⁰ M or less, or 1×10⁻¹⁰ M or less.

In some embodiments, the antibodies of the disclosure bind to cynomolgus monkey or mouse D3 at an EC₅₀ of no more than or about 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, 0.04 nM, 0.03 nM, 0.02 nM, or 0.01 nM, as measured by FACS.

### Anti-D3 Antibodies Comprising VHH CDRs

In some embodiments, an anti-D3 antibody as disclosed herein comprises at least one immunoglobulin single variable domain (e.g., VHH), wherein the VHH comprises CDR1, CDR2 and CDR3, and wherein CDR1 comprises an amino acid sequence as set forth in SEQ ID NO: 1, 4, 7 or 10, CDR2 comprises an amino acid sequence as set forth in SEQ ID NO: 2, 5, 8 or 11, and CDR3 comprises an amino acid sequence as set forth in SEQ ID NO: 3, 6 or 9. In some embodiments, the CDR numbering are according to a combination of Kabat and AbM numbering.

The extent of the framework region and CDRs can be precisely identified using methodology known in the art, for example, by the Kabat definition, the Chothia definition, the AbM definition, the contact definition, the IMGT definition (all of which are well known in the art) and any combinations thereof. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May, 63(1):78-85; and Martin and Allen, in “Handbook of Therapeutic Antibodies”, chapter 5, 2007. See also hgmp.mrc.ac.uk and bioinf.org.uk/abs. Correspondence or alignments between numberings according to different definitions can for example be found at www.imgt.org/ (see also Giudicelli V et al. IMGT, the international ImMunoGeneTics database. Nucleic Acids Res. (1997) 25:206-11; and Lefranc M P et al., IMGT unique numbering for immunoglobulin and T cell receptor variable domains and Ig superfamily V-like domains. Dev Comp Immunol. (2003) 27:55-77).

As will be appreciated by those in the art, the exact numbering and placement of the CDRs can be different among different numbering systems. However, it should be understood that the disclosure of a variable heavy sequence, a variable light sequence and/or a VHH sequence includes the disclosure of the associated (inherent) CDRs. Accordingly, the disclosure of each variable region is a disclosure of the CDRs (e.g., CDR1, CDR2 and CDR3). Two antibodies having the same VH, VL or VHH CDRs means that their CDRs are identical when determined by the same approach (e.g., the Kabat, AbM, Chothia, Contact, and IMGT numbering approaches as known in the art).

Variable regions and CDRs in an antibody sequence can be identified according to general rules that have been developed in the art (for example, the Kabat, AbM, Chothia, Contact, and IMGT numbering system) or by aligning the sequences against a database of known variable regions. Methods for identifying these regions are described in Kontermann and Dubel, eds., Antibody Engineering, Springer, New York, NY, 2001 and Dinarello et al., Current Protocols in Immunology, John Wiley and Sons Inc., Hoboken, NJ, 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005). Preferably sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. FIG. 10 shows an alignment of exemplary immunoglobulin single variable domains and boundaries of CDRs are indicated by Kabat, AbM, Chothia, Contact, and IMGT numbering.

In some embodiments, a D3-binding molecule as disclosed herein comprises at least one immunoglobulin single variable domain (e.g., VHH), wherein the VHH comprises FRW1-CDR1-FRW2-CDR2-FRW3-CDR3-FRW4, and wherein CDR1 has an amino acid sequence as set forth in SEQ ID NO: 1, 4, 7 or 10, CDR2 has an amino acid sequence as set forth in SEQ ID NO: 2, 5, 8 or 11, and CDR3 has an amino acid sequence as set forth in SEQ ID NO: 3, 6 or 9. In some embodiments, the FRW1 and FRW4 at the N and C terminal of the VHH comprised in a D3-binding molecule may be truncated such that it comprise only a partial FRW1 and/or FRW4, or the VHH lacks one or both of these framework regions, so long as the VHH substantially maintains antigen binding and specificity.

In some embodiments, provided herein is an anti-D3 antibody (such as an anti-D3 single domain antibody) comprising one, two, or all three CDRs of the amino acid sequence as set forth in SEQ ID NO: 12. In some embodiments, there is provided an anti-D3 antibody (such as an anti-D3 single domain antibody) comprising one, two, or all three CDRs of the amino acid sequence as set forth in SEQ ID NO: 13. In some embodiments, there is provided an anti-D3 antibody (such as an anti-D3 single domain antibody) comprising one, two, or all three CDRs of the amino acid sequence as set forth in SEQ ID NO: 14. In some embodiments, there is provided an anti-D3 antibody (such as an anti-D3 single domain antibody) comprising one, two, or all three CDRs of the amino acid sequence as set forth in SEQ ID NO: 15. In some embodiments, there is provided an anti-D3 antibody (such as an anti-D3 single domain antibody) comprising one, two, or all three CDRs of the amino acid sequence as set forth in SEQ ID NO: 16. In some embodiments, there is provided an anti-D3 antibody (such as an anti-D3 single domain antibody) comprising one, two, or all three CDRs of the amino acid sequence as set forth in SEQ ID NO: 17. In some embodiments, there is provided an anti-D3 antibody (such as an anti-D3 single domain antibody) comprising one, two, or all three CDRs of the amino acid sequence as set forth in SEQ ID NO: 18. In some embodiments, there is provided an anti-D3 antibody (such as an anti-D3 single domain antibody) comprising one, two, or all three CDRs of the amino acid sequence as set forth in SEQ ID NO: 55. In some embodiments, the anti-D3 single domain antibody is camelid. In some embodiments, the anti-D3 antibody (such as the anti-D3 single domain antibody) is humanized. In some embodiments, the anti-D3 antibody (such as the anti-D3 single domain antibody) comprises an acceptor human framework, e.g., a human immunoglobulin framework or a human consensus framework.

In some embodiments, the anti-D3 antibody (such as the single domain antibody) comprises a CDR1 having an amino acid sequence of the CDR1 as set forth in SEQ ID NO: 12. In some embodiments, the anti-D3 antibody (such as the single domain antibody) comprises a CDR2 having an amino acid sequence of the CDR2 as set forth in SEQ ID NO: 12. In other embodiments, the anti-D3 antibody (such as the single domain antibody) comprises a CDR3 having an amino acid sequence of the CDR3 as set forth in SEQ ID NO: 12. In some embodiments, the anti-D3 antibody (such as the single domain antibody) comprises a CDR1 and a CDR2 having amino acid sequences of the CDR1 and the CDR2 as set forth in SEQ ID NO: 12. In some embodiments, the anti-D3 antibody (such as the single domain antibody) comprises a CDR1 and a CDR3 having amino acid sequences of the CDR1 and the CDR3 as set forth in SEQ ID NO: 12. In some embodiments, the anti-D3 antibody (such as the single domain antibody) comprises a CDR2 and a CDR3 having amino acid sequences of the CDR2 and the CDR3 as set forth in SEQ ID NO: 12. In some embodiments, the anti-D3 antibody (such as the single domain antibody) comprises a CDR1, a CDR2, and a CDR3 having amino acid sequences of the CDR1, the CDR2, and the CDR3 as set forth in SEQ ID NO: 12. CDR sequences can be determined according to well-known numbering systems. In some embodiments, the CDRs are according to IMGT numbering. In some embodiments, the CDRs are according to Kabat numbering. In other embodiments, the CDRs are according to Chothia numbering. In other embodiments, the CDRs are according to Contact numbering. In some embodiments, the CDRs are according to AbM numbering. In some embodiments, the anti-D3 single domain antibody is camelid. In some embodiments, the anti-D3 antibody (such as the anti-D3 single domain antibody) is humanized. In some embodiments, the anti-D3 antibody (such as the anti-D3 single domain antibody) comprises an acceptor human framework, e.g., a human immunoglobulin framework or a human consensus framework.

In some embodiments, the single domain antibody has a CDR1 having an amino acid sequence of the CDR1 as set forth in SEQ ID NO: 13. In some embodiments, the single domain antibody has a CDR2 having an amino acid sequence of the CDR2 as set forth in SEQ ID NO: 13. In other embodiments, the single domain antibody has a CDR3 having an amino acid sequence of the CDR3 as set forth in SEQ ID NO: 13. In some embodiments, the single domain antibody has a CDR1 and a CDR2 having amino acid sequences of the CDR1 and the CDR2 as set forth in SEQ ID NO: 13. In some embodiments, the single domain antibody has a CDR1 and a CDR3 having amino acid sequences of the CDR1 and the CDR3 as set forth in SEQ ID NO: 13. In some embodiments, the single domain antibody has a CDR2 and a CDR3 having amino acid sequences of the CDR2 and the CDR3 as set forth in SEQ ID NO: 13. In some embodiments, the single domain antibody has a CDR1, a CDR2, and a CDR3 having amino acid sequences of the CDR1, the CDR2, and the CDR3 as set forth in SEQ ID NO: 13. CDR sequences can be determined according to well-known numbering systems. In some embodiments, the CDRs are according to IMGT numbering. In some embodiments, the CDRs are according to Kabat numbering. In other embodiments, the CDRs are according to Chothia numbering. In other embodiments, the CDRs are according to Contact numbering. In some embodiments, the CDRs are according to AbM numbering. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 14. In some embodiments, the single domain antibody has a CDR2 having an amino acid sequence of the CDR2 as set forth in SEQ ID NO: 14. In other embodiments, the single domain antibody has a CDR3 having an amino acid sequence of the CDR3 as set forth in SEQ ID NO: 14. In some embodiments, the single domain antibody has a CDR1 and a CDR2 having amino acid sequences of the CDR1 and the CDR2 as set forth in SEQ ID NO: 14. In some embodiments, the single domain antibody has a CDR1 and a CDR3 having amino acid sequences of the CDR1 and the CDR3 as set forth in SEQ ID NO: 14. In some embodiments, the single domain antibody has a CDR2 and a CDR3 having amino acid sequences of the CDR2 and the CDR3 as set forth in SEQ ID NO: 14. In some embodiments, the single domain antibody has a CDR1, a CDR2, and a CDR3 having amino acid sequences of the CDR1, the CDR2, and the CDR3 as set forth in SEQ ID NO: 14. CDR sequences can be determined according to well-known numbering systems. In some embodiments, the CDRs are according to IMGT numbering. In some embodiments, the CDRs are according to Kabat numbering. In other embodiments, the CDRs are according to Chothia numbering. In other embodiments, the CDRs are according to Contact numbering. In some embodiments, the CDRs are according to AbM numbering. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 15. In some embodiments, the single domain antibody has a CDR2 having an amino acid sequence of the CDR2 as set forth in SEQ ID NO: 15. In other embodiments, the single domain antibody has a CDR3 having an amino acid sequence of the CDR3 as set forth in SEQ ID NO: 15. In some embodiments, the single domain antibody has a CDR1 and a CDR2 having amino acid sequences of the CDR1 and the CDR2 as set forth in SEQ ID NO: 15. In some embodiments, the single domain antibody has a CDR1 and a CDR3 having amino acid sequences of the CDR1 and the CDR3 as set forth in SEQ ID NO: 15. In some embodiments, the single domain antibody has a CDR2 and a CDR3 having amino acid sequences of the CDR2 and the CDR3 as set forth in SEQ ID NO: 15. In some embodiments, the single domain antibody has a CDR1, a CDR2, and a CDR3 having amino acid sequences of the CDR1, the CDR2, and the CDR3 as set forth in SEQ ID NO: 15. CDR sequences can be determined according to well-known numbering systems. In some embodiments, the CDRs are according to IMGT numbering. In some embodiments, the CDRs are according to Kabat numbering. In other embodiments, the CDRs are according to Chothia numbering. In other embodiments, the CDRs are according to Contact numbering. In some embodiments, the CDRs are according to AbM numbering. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 16. In some embodiments, the single domain antibody has a CDR2 having an amino acid sequence of the CDR2 as set forth in SEQ ID NO: 16. In other embodiments, the single domain antibody has a CDR3 having an amino acid sequence of the CDR3 as set forth in SEQ ID NO: 16. In some embodiments, the single domain antibody has a CDR1 and a CDR2 having amino acid sequences of the CDR1 and the CDR2 as set forth in SEQ ID NO: 16. In some embodiments, the single domain antibody has a CDR1 and a CDR3 having amino acid sequences of the CDR1 and the CDR3 as set forth in SEQ ID NO: 16. In some embodiments, the single domain antibody has a CDR2 and a CDR3 having amino acid sequences of the CDR2 and the CDR3 as set forth in SEQ ID NO: 16. In some embodiments, the single domain antibody has a CDR1, a CDR2, and a CDR3 having amino acid sequences of the CDR1, the CDR2, and the CDR3 as set forth in SEQ ID NO: 16. CDR sequences can be determined according to well-known numbering systems. In some embodiments, the CDRs are according to IMGT numbering. In some embodiments, the CDRs are according to Kabat numbering. In other embodiments, the CDRs are according to Chothia numbering. In other embodiments, the CDRs are according to Contact numbering. In some embodiments, the CDRs are according to AbM numbering. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 17. In some embodiments, the single domain antibody has a CDR2 having an amino acid sequence of the CDR2 as set forth in SEQ ID NO: 17. In other embodiments, the single domain antibody has a CDR3 having an amino acid sequence of the CDR3 as set forth in SEQ ID NO: 17. In some embodiments, the single domain antibody has a CDR1 and a CDR2 having amino acid sequences of the CDR1 and the CDR2 as set forth in SEQ ID NO: 17. In some embodiments, the single domain antibody has a CDR1 and a CDR3 having amino acid sequences of the CDR1 and the CDR3 as set forth in SEQ ID NO: 17. In some embodiments, the single domain antibody has a CDR2 and a CDR3 having amino acid sequences of the CDR2 and the CDR3 as set forth in SEQ ID NO: 17. In some embodiments, the single domain antibody has a CDR1, a CDR2, and a CDR3 having amino acid sequences of the CDR1, the CDR2, and the CDR3 as set forth in SEQ ID NO: 17. CDR sequences can be determined according to well-known numbering systems. In some embodiments, the CDRs are according to IMGT numbering. In some embodiments, the CDRs are according to Kabat numbering. In other embodiments, the CDRs are according to Chothia numbering. In other embodiments, the CDRs are according to Contact numbering. In some embodiments, the CDRs are according to AbM numbering. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the single domain antibody has a CDR1 having an amino acid sequence of the CDR1 as set forth in SEQ ID NO: 18. In some embodiments, the single domain antibody has a CDR2 having an amino acid sequence of the CDR2 as set forth in SEQ ID NO: 18. In other embodiments, the single domain antibody has a CDR3 having an amino acid sequence of the CDR3 as set forth in SEQ ID NO: 18. In some embodiments, the single domain antibody has a CDR1 and a CDR2 having amino acid sequences of the CDR1 and the CDR2 as set forth in SEQ ID NO: 18. In some embodiments, the single domain antibody has a CDR1 and a CDR3 having amino acid sequences of the CDR1 and the CDR3 as set forth in SEQ ID NO: 18. In some embodiments, the single domain antibody has a CDR2 and a CDR3 having amino acid sequences of the CDR2 and the CDR3 as set forth in SEQ ID NO: 18. In some embodiments, the single domain antibody has a CDR1, a CDR2, and a CDR3 having amino acid sequences of the CDR1, the CDR2, and the CDR3 as set forth in SEQ ID NO: 18. CDR sequences can be determined according to well-known numbering systems. In some embodiments, the CDRs are according to IMGT numbering. In some embodiments, the CDRs are according to Kabat numbering. In other embodiments, the CDRs are according to Chothia numbering. In other embodiments, the CDRs are according to Contact numbering. In some embodiments, the CDRs are according to AbM numbering. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, provided herein is a single domain antibody that binds to D3 comprising the following structure: FR1-CDR1-FR2-CDR2-FR3-CDR3-FR4, wherein (i) the CDR1 comprises an amino acid sequence as set forth in SEQ ID NO: 1, SEQ ID NO: 4, SEQ ID NO: 7, SEQ ID NO: 10, SEQ ID NO: 20, SEQ ID NO: 23, SEQ ID NO: 24, SEQ ID NO: 27, SEQ ID NO: 31, SEQ ID NO: 34, SEQ ID NO: 35, SEQ ID NO: 38, SEQ ID NO: 42, SEQ ID NO: 45, SEQ ID NO: 46, SEQ ID NO: 49, SEQ ID NO: 53, or SEQ ID NO: 54; (ii) the CDR2 comprises an amino acid sequence as set forth in SEQ ID NO: 2, SEQ ID NO: 5, SEQ ID NO: 8, SEQ ID NO: 11, SEQ ID NO: 21, SEQ ID NO: 25, SEQ ID NO: 28, SEQ ID NO: 56, SEQ ID NO: 30, SEQ ID NO: 32, SEQ ID NO: 36, SEQ ID NO: 39, SEQ ID NO: 41, SEQ ID NO: 43, SEQ ID NO: 47, SEQ ID NO: 50, or SEQ ID NO: 52; and/or (iii) the CDR3 comprises an amino acid sequence as set forth in SEQ ID NO: 3, SEQ ID NO: 6, SEQ ID NO: 9, SEQ ID NO: 22, SEQ ID NO: 26, SEQ ID NO: 29, SEQ ID NO: 33, SEQ ID NO: 37, SEQ ID NO: 40, SEQ ID NO: 44, SEQ ID NO: 48, or SEQ ID NO: 51. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the CDR1 comprises the exemplary amino acid sequence as set forth in SEQ ID NO: 1; the CDR2 comprises the exemplary amino acid sequence as set forth in SEQ ID NO: 2; and the CDR3 comprises the exemplary amino acid sequence as set forth in SEQ ID NO: 3. In some embodiments, the CDR1 is according to IMGT numbering, comprising the amino acid sequence as set forth in SEQ ID NO: 20; the CDR2 is according to IMGT numbering, comprising the amino acid sequence as set forth in SEQ ID NO: 21; and the CDR3 is according to IMGT numbering, comprising the amino acid sequence as set forth in SEQ ID NO: 22. In some embodiments, the CDR1 is according to Kabat numbering, comprising the amino acid sequence as set forth in SEQ ID NO: 23; the CDR2 is according to Kabat numbering, comprising the amino acid sequence as set forth in SEQ ID NO: 2; and the CDR3 is according to Kabat numbering, comprising the amino acid sequence as set forth in SEQ ID NO: 3. In some embodiments, the CDR1 is according to Chothia numbering, comprising the amino acid sequence as set forth in SEQ ID NO: 24; the CDR2 is according to Chothia numbering, comprising the amino acid sequence as set forth in SEQ ID NO: 25; and the CDR3 is according to Chothia numbering, comprising the amino acid sequence as set forth in SEQ ID NO: 26. In some embodiments, the CDR1 is according to Contact numbering, comprising the amino acid sequence as set forth in SEQ ID NO: 27; the CDR2 is according to Contact numbering, comprising the amino acid sequence as set forth in SEQ ID NO: 28 or 56; and the CDR3 is according to Contact numbering, comprising the amino acid sequence as set forth in SEQ ID NO: 29. In some embodiments, the CDR1 is according to AbM numbering, comprising the amino acid sequence as set forth in SEQ ID NO: 1; the CDR2 is according to AbM numbering, comprising the amino acid sequence as set forth in SEQ ID NO: 30; and the CDR3 is according to AbM numbering, comprising the amino acid sequence as set forth in SEQ ID NO: 3. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the CDR1 comprises the exemplary amino acid sequence as set forth in SEQ ID NO: 4; the CDR2 comprises the exemplary amino acid sequence as set forth in SEQ ID NO: 5; and the CDR3 comprises the exemplary amino acid sequence as set forth in SEQ ID NO: 6. In some embodiments, the CDR1 is according to IMGT numbering, comprising the amino acid sequence as set forth in SEQ ID NO: 31; the CDR2 is according to IMGT numbering, comprising the amino acid sequence as set forth in SEQ ID NO: 32; and the CDR3 is according to IMGT numbering, comprising the amino acid sequence as set forth in SEQ ID NO: 33. In some embodiments, the CDR1 is according to Kabat numbering, comprising the amino acid sequence as set forth in SEQ ID NO: 34; the CDR2 is according to Kabat numbering, comprising the amino acid sequence as set forth in SEQ ID NO: 5; and the CDR3 is according to Kabat numbering, comprising the amino acid sequence as set forth in SEQ ID NO: 6. In some embodiments, the CDR1 is according to Chothia numbering, comprising the amino acid sequence as set forth in SEQ ID NO: 35; the CDR2 is according to Chothia numbering, comprising the amino acid sequence as set forth in SEQ ID NO: 36; and the CDR3 is according to Chothia numbering, comprising the amino acid sequence as set forth in SEQ ID NO: 37. In some embodiments, the CDR1 is according to Contact numbering, comprising the amino acid sequence as set forth in SEQ ID NO: 38; the CDR2 is according to Contact numbering, comprising the amino acid sequence as set forth in SEQ ID NO: 39; and the CDR3 is according to Contact numbering, comprising the amino acid sequence as set forth in SEQ ID NO: 40. In some embodiments, the CDR1 is according to AbM numbering, comprising the amino acid sequence as set forth in SEQ ID NO: 4; the CDR2 is according to AbM numbering, comprising the amino acid sequence as set forth in SEQ ID NO: 41; and the CDR3 is according to AbM numbering, comprising the amino acid sequence as set forth in SEQ ID NO: 6. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the CDR1 comprises the exemplary amino acid sequence as set forth in SEQ ID NO: 7; the CDR2 comprises the exemplary amino acid sequence as set forth in SEQ ID NO: 8; and the CDR3 comprises the exemplary amino acid sequence as set forth in SEQ ID NO: 9. In some embodiments, the CDR1 is according to IMGT numbering, comprising the amino acid sequence as set forth in SEQ ID NO: 42; the CDR2 is according to IMGT numbering, comprising the amino acid sequence as set forth in SEQ ID NO: 43; and the CDR3 is according to IMGT numbering, comprising the amino acid sequence as set forth in SEQ ID NO: 44. In some embodiments, the CDR1 is according to Kabat numbering, comprising the amino acid sequence as set forth in SEQ ID NO: 45; the CDR2 is according to Kabat numbering, comprising the amino acid sequence as set forth in SEQ ID NO: 8; and the CDR3 is according to Kabat numbering, comprising the amino acid sequence as set forth in SEQ ID NO: 9. In some embodiments, the CDR1 is according to Chothia numbering, comprising the amino acid sequence as set forth in SEQ ID NO: 46; the CDR2 is according to Chothia numbering, comprising the amino acid sequence as set forth in SEQ ID NO: 47; and the CDR3 is according to Chothia numbering, comprising the amino acid sequence as set forth in SEQ ID NO: 48. In some embodiments, the CDR1 is according to Contact numbering, comprising the amino acid sequence as set forth in SEQ ID NO: 49, the CDR2 is according to Contact numbering, comprising the amino acid sequence as set forth in SEQ ID NO: 50; and the CDR3 is according to Contact numbering, comprising the amino acid sequence as set forth in SEQ ID NO: 51. In some embodiments, the CDR1 is according to AbM numbering, comprising the amino acid sequence as set forth in SEQ ID NO: 7; the CDR2 is according to AbM numbering, comprising the amino acid sequence as set forth in SEQ ID NO: 52; and the CDR3 is according to AbM numbering, comprising the amino acid sequence as set forth in SEQ ID NO: 9. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the CDR1 comprises the exemplary amino acid sequence as set forth in SEQ ID NO: 10; the CDR2 comprises the exemplary amino acid sequence as set forth in SEQ ID NO: 11; and the CDR3 comprises the exemplary amino acid sequence as set forth in SEQ ID NO: 6. In some embodiments, the CDR1 is according to IMGT numbering, comprising the amino acid sequence as set forth in SEQ ID NO: 53; the CDR2 is according to IMGT numbering, comprising the amino acid sequence as set forth in SEQ ID NO: 32; and the CDR3 is according to IMGT numbering, comprising the amino acid sequence as set forth in SEQ ID NO: 33. In some embodiments, the CDR1 is according to Kabat numbering, comprising the amino acid sequence as set forth in SEQ ID NO: 34; the CDR2 is according to Kabat numbering, comprising the amino acid sequence as set forth in SEQ ID NO: 11; and the CDR3 is according to Kabat numbering, comprising the amino acid sequence as set forth in SEQ ID NO: 6. In some embodiments, the CDR1 is according to Chothia numbering, comprising the amino acid sequence as set forth in SEQ ID NO: 54; the CDR2 is according to Chothia numbering, comprising the amino acid sequence as set forth in SEQ ID NO: 36; and the CDR3 is according to Chothia numbering, comprising the amino acid sequence as set forth in SEQ ID NO: 37. In some embodiments, the CDR1 is according to Contact numbering, comprising the amino acid sequence as set forth in SEQ ID NO: 38; the CDR2 is according to Contact numbering, comprising the amino acid sequence as set forth in SEQ ID NO: 39; and the CDR3 is according to Contact numbering, comprising the amino acid sequence as set forth in SEQ ID NO: 40. In some embodiments, the CDR1 is according to AbM numbering, comprising the amino acid sequence as set forth in SEQ ID NO: 10; the CDR2 is according to AbM numbering, comprising the amino acid sequence as set forth in SEQ ID NO: 41; and the CDR3 is according to AbM numbering, comprising the amino acid sequence as set forth in SEQ ID NO: 6. In some embodiments, the anti-D3 single domain antibody is camelid. In some embodiments, the anti-D3 single domain antibody is humanized. In some embodiments, the anti-D3 single domain antibody comprises an acceptor human framework, e.g., a human immunoglobulin framework or a human consensus framework.

In some embodiments, the single domain antibody further comprises one or more framework regions of WT1156-P3R2-1C2, WT1156-P3R2-1C9, WT1156-P8R2-1H1, WT1156-P3R2-1H6, WT1156-P3R2-1C2-z102, WT1156-P3R2-1C2-z109, and/or WT1156-P3R2-1H6-z100. In some embodiments, the single domain antibody comprises one or more framework(s) derived from a VHH domain comprising the sequence as set forth in SEQ ID NO: 12. In some embodiments, the single domain antibody comprises one or more framework(s) derived from a VHH domain comprising the sequence as set forth in SEQ ID NO: 13. In some embodiments, the single domain antibody comprises one or more framework(s) derived from a VHH domain comprising the sequence as set forth in SEQ ID NO: 14. In some embodiments, the single domain antibody comprises one or more framework(s) derived from a VHH domain comprising the sequence as set forth in SEQ ID NO: 15. In some embodiments, the single domain antibody comprises one or more framework(s) derived from a VHH domain comprising the sequence as set forth in SEQ ID NO: 16. In some embodiments, the single domain antibody comprises one or more framework(s) derived from a VHH domain comprising the sequence as set forth in SEQ ID NO: 17. In some embodiments, the single domain antibody comprises one or more framework(s) derived from a VHH domain comprising the sequence as set forth in SEQ ID NO: 18. In some embodiments, the single domain antibody comprises one or more framework(s) derived from a VHH domain comprising the sequence as set forth in SEQ ID NO: 55.

In some embodiments, the single domain antibody provided herein is a humanized single domain antibody.

Framework regions described herein are determined based upon the boundaries of the CDR numbering system. In other words, if the CDRs are determined by, e.g., IMGT, Kabat, Chothia, Contact, or AbM, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the IMGT numbering system, the Kabat numbering system, the Chothia numbering system, the Contact numbering system, or the AbM numbering system, FR2 is defined as the amino acid residues between CDR1 and CDR2 amino acid residues as defined by, e.g., the IMGT numbering system, the Kabat numbering system, the Chothia numbering system, the Contact numbering system, or the AbM numbering system, FR3 is defined as the amino acid residues between CDR2 and CDR3 amino acid residues as defined by, e.g., the IMGT numbering system, the Kabat numbering system, the Chothia numbering system, the Contact numbering system, or the AbM numbering system, and FR4 is defined as the amino acid residues C-terminal to the CDR3 amino acid residues as defined by, e.g., the IMGT numbering system, the Kabat numbering system, the Chothia numbering system, the Contact numbering system, or the AbM numbering system.

In some embodiments, there is provided an isolated anti-D3 single domain antibody comprising a VHH domain having the amino acid sequence as set forth in SEQ ID NO: 12. In some embodiments, there is provided a polypeptide comprising the amino acid sequence as set forth in SEQ ID NO: 12. In some embodiments, there is provided an isolated anti-D3 single domain antibody comprising a VHH domain having the amino acid sequence as set forth in SEQ ID NO: 13. In some embodiments, there is provided a polypeptide comprising the amino acid sequence as set forth in SEQ ID NO: 13. In some embodiments, there is provided an isolated anti-D3 single domain antibody comprising a VHH domain having the amino acid sequence as set forth in SEQ ID NO: 14. In some embodiments, there is provided a polypeptide comprising the amino acid sequence as set forth in SEQ ID NO: 14. In some embodiments, there is provided an isolated anti-D3 single domain antibody comprising a VHH domain having the amino acid sequence as set forth in SEQ ID NO: 15. In some embodiments, there is provided a polypeptide comprising the amino acid sequence as set forth in SEQ ID NO: 15. In some embodiments, there is provided an isolated anti-D3 single domain antibody comprising a VHH domain having the amino acid sequence as set forth in SEQ ID NO: 16. In some embodiments, there is provided a polypeptide comprising the amino acid sequence as set forth in SEQ ID NO: 16. In some embodiments, there is provided an isolated anti-D3 single domain antibody comprising a VHH domain having the amino acid sequence as set forth in SEQ ID NO: 17. In some embodiments, there is provided a polypeptide comprising the amino acid sequence as set forth in SEQ ID NO: 17. In some embodiments, there is provided an isolated anti-D3 single domain antibody comprising a VHH domain having the amino acid sequence as set forth in SEQ ID NO: 18. In some embodiments, there is provided a polypeptide comprising the amino acid sequence as set forth in SEQ ID NO: 18. In some embodiments, there is provided an isolated anti-D3 single domain antibody comprising a VHH domain having the amino acid sequence as set forth in SEQ ID NO: 55. In some embodiments, there is provided a polypeptide comprising the amino acid sequence as set forth in SEQ ID NO: 55.

### Anti-D3 Antibodies Comprising VHH Sequences

In some embodiments, anti-D3 antibodies comprise at least one immunoglobulin single variable domain (e.g., VHH), wherein the VHH comprises or consists of:
(A) an amino acid sequence as set forth in any one of SEQ ID NOs: 12-18 and 55;
(B) an amino acid sequence which is at least 85%, at least 90%, or at least 95% identical to any one of SEQ ID NOs: 12-18 and 55; or
(C) an amino acid sequence with addition, deletion and/or substitution of one or more (for example, 1, 2, 3, 4, 5, 6, 7, 8, 9 or 10) amino acids compared with any one of SEQ ID NOs: 12-18 and 55.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, protein (e.g., antibody) sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs {e.g., XBLAST and NBLAST) can be used. See www.ncbi.nlm.nih.gov.

In some embodiments, the amino acid sequence of a VHH can be at least 85%, 86%, 87%, 88%, 89%, 90%, 91%, 92%, 93%, 94%, 95%, 96%, 97%, 98% or 99% identical to any one of SEQ ID NOs: 12-18 and 55.

In some further embodiments, anti-D3 antibodies may contain conservative substitution or modification of amino acids in the variable regions and/or constant regions. It is understood in the art that certain conservative sequence modification can be made which do not remove antigen binding. See, e.g., Brummell et al. (1993) Biochem 32:1180-8; de Wildt et al. (1997) Prot. Eng. 10:835-41; Komissarov et al. (1997) J. Biol. Chem. 272:26864-26870; Hall et al. (1992) J. Immunol. 149:1605-12; Kelley and O'Connell (1993) Biochem. 32:6862-35; Adib-Conquy et al. (1998) Int. Immunol. 10:341-6 and Beers et al. (2000) Clin. Can. Res. 6:2835-43.

As described above, the term “conservative substitution”, as used herein, refers to an amino acid substitution which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

In some embodiments, an anti-D3 antibody comprises at least one VHH, and the VHH comprises an amino acid sequence as set forth in any one of SEQ ID NOs: 12-18. In some embodiments, the anti-D3 antibody comprises a VHH which has the amino acid sequence as set forth in any one of SEQ ID NOs: 12-18.

In some embodiments, an anti-D3 antibody is a chimeric antibody, comprising a VHH fused to an Fc region of human IgG1 or IgG4, wherein the VHH comprises an amino acid sequence as set forth in any one of SEQ ID NOs: 12-18 and 55. In some embodiments, an anti-D3 antibody is a chimeric antibody comprising a VHH and an Fc region of human IgG1. Such antibodies are exemplified herein as “WT1156-P3R2-1C2-uIgG1”, “WT1156-P3R2-1H6-uIgG1”, “WT1156-P8R2-1H1-uIgG1”, and “WT1156-P3R2-1C9-uIgG1”. In some further embodiments, an anti-D3 antibody is a humanized antibody comprising a VHH and an Fc region of human IgG1. Such antibodies are exemplified herein as “WT1156-P3R2-1C2-z102-uIgG1”, “WT1156-P3R2-1C2-z109-uIgG1” and “WT1156-P3R2-1H6-z100-uIgG1”.

In some embodiments, the addition, deletion and/or substitution of at least one of the amino acids in the VHH region is not in any of the CDR sequences, but in the framework (FRW) sequences. For example, an antibody or antigen-binding portion thereof as described above may comprise one or more substitutions of the amino acids in the framework sequences, e.g. FRW1, FRW2, FRW3, and/or FRW4 of the VHH region.

In some embodiments, an antibody or antigen-binding portion thereof as provided herein comprises any suitable framework region (FRW) sequences, as long as the antigen-binding domains can specifically bind to D3.

As described above, an antibody or antigen-binding portion thereof may contain modification of one or more amino acids in the variable regions of the heavy chain and/or light chain, including wherein the modification is a conservative substitution. It is understood in the art that certain conservative sequence modifications can be made which do not remove antigen binding. See, e.g., Brummell et al. (1993) Biochem 32:1180-8; de Wildt et al. (1997) Prot. Eng. 10:835-41; Komissarov et al. (1997) J. Biol. Chem. 272:26864-26870; Hall et al. (1992) J. Immunol. 149:1605-12; Kelley and O'Connell (1993) Biochem. 32:6862-35; Adib-Conquy et al. (1998) Int. Immunol. 10:341-6 and Beers et al. (2000) Clin. Can. Res. 6:2835-43.

In some embodiments, an antibody or antigen-binding portion thereof comprises a VHH domain comprising an amino acid sequence as set forth in any one of SEQ ID NOs: 12-18 and 55, and a Fc region comprising an amino acid sequence as set forth in SEQ ID NO: 19.

An antigen-binding domain of a D3-binding molecule is not limited to the VHH form and may adopt a variety of other formats, such as but not limited to, a Fab, a Fab′, a F(ab′)₂, an Fv fragment, a single-chain antibody molecule (scFv). In some embodiments, an antigen-binding domain is a Fv fragment with a VH region and a VL region in separate chains held together by tight, non-covalent interactions.

### Fc Region Comprising IgG Constant Domains

Anti-D3 antibodies and antigen-binding fragments provided herein further comprise an Fc region comprising one or more human IgG constant domains. A human IgG constant domain may be a human IgG1, IgG2, IgG3 or IgG4 constant domain, preferably a human IgG1 constant domain. An example of the amino acid sequence of an Fc region comprising human IgG1 constant regions is set forth in SEQ ID NO: 19. In some embodiments, the Fc region is a human IgG1 Fc region, such as a wild-type Fc region or a Fc variant comprising one or more amino acid modifications (e.g. Leu234Ala/Leu235Ala or LALA) that alters the antibody-dependent cellular cytotoxicity (ADCC) or other effector functions.

In some embodiments, the Fc modification comprises a LALA mutation, e.g. mutations of L234A and L235A, according to EU numbering as in Kabat et al., The Kabat numbering system is often used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU numbering as in Kabat” or “EU index as in Kabat” refers to the residue numbering of the human IgG1 EU antibody. Unless stated otherwise herein, references to residue numbers in the constant domain of antibodies means residue numbering by the EU numbering system.

### Nucleic Acid Molecules Encoding Antibodies of the Disclosure

In some aspects, the present disclosure provides a nucleic acid molecule comprising a nucleic acid sequence encoding a D3-binding molecule as disclosed herein, for example, encoding a single variable domain of a D3-binding molecule as disclosed herein. Nucleic acids of the disclosure can be obtained using standard molecular biology techniques.

A nucleic acid encoding a VHH region can be converted to a full-length heavy chain gene by operatively linking the VHH-encoding nucleic acid to another nucleic acid encoding one or more heavy chain constant regions (e.g. CH1, CH2 and CH3). The sequences of human heavy chain constant region genes are known in the art (see e.g., Kabat et al. (1991), supra) and DNA fragments encompassing these regions can be obtained by standard PCR amplification. A heavy chain constant region can be an IgG1, IgG2, IgG3, IgG4, IgA, IgE, IgM or IgD constant region, such as an IgG1 constant region.

Once nucleic acids encoding VHH segments are obtained, these nucleic acids can be further manipulated by standard recombinant DNA techniques, for example to convert variable region genes to full-length antibody chain genes. In these manipulations, a VHH-encoding nucleic acid is operatively linked to another nucleic acid encoding another protein, such as an antibody constant region or a flexible linker. The term “operatively linked”, as used in this context, is intended to mean that two or more nucleic acids are joined such that the amino acid sequences encoded by the two or more nucleic acids remain in-frame.

In some embodiments, the disclosure is directed to a nucleic acid molecule, comprising a nucleic acid sequence encoding a single variable domain (e.g., VHH) of a D3-binding molecule as disclosed herein.

In some embodiments, the nucleic acid molecule comprises a nucleic acid sequence selected from the group consisting of:
(A) a nucleic acid sequence that encodes a VHH region as set forth in any one of SEQ ID NOs: 12-18 and 55;
(B) a nucleic acid sequence with at least 80% (e.g. at least 85%, 86%, 87%, 88%, 89%, 90%, 91%, 92%, 93%, 94%, 95%, 96%, 97%, 98%, or 99%) sequence identity to the nucleic acid sequence of (A); and
(C) a nucleic acid sequence that hybridizes under high stringency conditions to the complementary strand of the nucleic acid sequence of (A).

In some embodiments, provided herein is a nucleic acid molecule comprises a nucleic acid sequence encoding an anti-D3 single domain antibody comprising the amino acid sequence as set forth in SEQ ID NO: 12. In some embodiments, provided herein is a nucleic acid molecule comprises a nucleic acid sequence encoding an anti-D3 single domain antibody comprising the amino acid sequence as set forth in SEQ ID NO: 13. In some embodiments, provided herein is a nucleic acid molecule comprises a nucleic acid sequence encoding an anti-D3 single domain antibody comprising the amino acid sequence as set forth in SEQ ID NO: 14. In some embodiments, provided herein is a nucleic acid molecule comprises a nucleic acid sequence encoding an anti-D3 single domain antibody comprising the amino acid sequence as set forth in SEQ ID NO: 15. In some embodiments, provided herein is a nucleic acid molecule comprises a nucleic acid sequence encoding an anti-D3 single domain antibody comprising the amino acid sequence as set forth in SEQ ID NO: 16. In some embodiments, provided herein is a nucleic acid molecule comprises a nucleic acid sequence encoding an anti-D3 single domain antibody comprising the amino acid sequence as set forth in SEQ ID NO: 17. In some embodiments, provided herein is a nucleic acid molecule comprises a nucleic acid sequence encoding an anti-D3 single domain antibody comprising the amino acid sequence as set forth in SEQ ID NO: 18. In some embodiments, provided herein is a nucleic acid molecule comprises a nucleic acid sequence encoding an anti-D3 single domain antibody comprising the amino acid sequence as set forth in SEQ ID NO: 55.

In some embodiments, the percent identity is derived from the degeneracy of the genetic code, and the encoded protein sequences remain unchanged.

Exemplary high stringency conditions include hybridization at 45° C. in 5×SSPE and 45% formamide, and a final wash at 65° C. in 0.1×SSC. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al, (Eds.), Molecular Cloning: A laboratory Manual. Cold Spring Harbor Laboratory Press: Cold Spring Harbor, New York (1989), pp. 9.47 to 9.51.

### Host Cells

Host cells as disclosed in the present disclosure may be any cell which is suitable for expressing the antibodies of the present disclosure, for example, yeast, bacterial, plant and mammalian cells. Mammalian host cells for expressing the antibodies of the present disclosure include Chinese Hamster Ovary (CHO cells) (including dhfr CHO cells, described in Urlaub and Chasin, (1980) Proc. Natl. Acad. ScL USA 77:4216-4220, used with a DHFR selectable marker, e.g., as described in R. J. Kaufman and P. A. Sharp (1982) J. MoI. Biol. 159:601-621), 293F cells, NSO myeloma cells, COS cells and SP2 cells. In particular, for use with NSO myeloma cells, another expression system is the GS gene expression system disclosed in WO 87/04462, WO 89/01036 and EP 338,841. Also included are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., 1980, Proc. Natl. Acad. Sci. USA 77:4216); mouse sertoli cells (TM4, Mather, 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; mouse myeloma cells, such as NSO (e.g. RCB0213, 1992, Bio/Technology 10:169) and SP2/0 cells (e.g. SP2/0-Agl4 cells, ATCC CRL 1581); rat myeloma cells, such as YB2/0 cells (e.g. YB2/3HL.P2.G11.16Ag.20 cells, ATCC CRL 1662); PER.C6 cells; and a human hepatoma line (Hep G2). CHO cells are one of the cell lines that can be used herein, with CHO-K1, DUK-B11, CHO-DP12, CHO-DG44 (Somatic Cell and Molecular Genetics 12:555 (1986)), and Lecl3 being exemplary host cell lines. In the case of CHO-K1, DUK-B11, DG44 or CHO-DP12 host cells, these may be altered such that they are deficient in their ability to fucosylate proteins expressed therein. In some embodiments, the host cells herein are selected from CHO, CHO-S, HEK, HEK293, HEK-293F, Expi293F, PER.C6 or NSO cells or lymphocytic cells.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are also suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurosporacrassa; Schwanniomyces such as Schwanniomycesoccidentalis; and filamentous fungi such as, e.g., Neurospora, Penicillium, Tolypocladium, and Aspergillus hosts such as A. nidulans and A. niger.

When recombinant expression vectors encoding an antibody are introduced into mammalian host cells, the antibody is produced by culturing the host cells for a period of time sufficient to allow for expression of the antibody in the host cells or, secretion of the antibody into the culture medium in which the host cells are grown. Antibodies can be recovered from the culture medium using standard protein purification methods.

### Pharmaceutical Compositions

In some aspects, the present disclosure provides a pharmaceutical composition comprising a D3-binding molecule as disclosed herein, for example, comprising a single variable domain (e.g., VHH) of a D3-binding molecule as disclosed herein and a pharmaceutically acceptable carrier. In some aspects, the present disclosure provides a pharmaceutical composition comprising a nucleic acid encoding a D3-binding molecule as disclosed herein, for example, comprising a single variable domain (e.g., VHH) of a D3-binding molecule as disclosed herein and a pharmaceutically acceptable carrier. In some aspects, the present disclosure provides a pharmaceutical composition comprising a cell expressing a D3-binding molecule as disclosed herein, for example, comprising a single variable domain (e.g., VHH) of a D3-binding molecule as disclosed herein and a pharmaceutically acceptable carrier.

### Components of the Compositions

The pharmaceutical composition may optionally contain one or more additional components, including one or more pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, including wherein the anti-D3 antibody enhances the immune response. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components of the pharmaceutical composition may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propyl gallate. As disclosed in the present disclosure, a composition may comprise an antibody or an antigen-binding fragment of the present disclosure and also comprise one or more anti-oxidants such as methionine, to prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

A pharmaceutical composition of the disclosure may be administered to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, a D3-binding molecule of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose; and any values within the ranges. Other ranges include about 100 μg/kg body weight to about 20 mg/kg body weight per dose and about 0.5 mg/kg body weight to about 20 mg/kg body weight per dose. In some embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, an antibody or antigen binding portion thereof of the disclosure is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In some embodiments, the course of treatment involving a D3-binding molecule of the present disclosure will comprise multiple doses of the selected drug product over a period of weeks or months. For example, a D3-binding molecule of the present disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In some embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) may comprise a D3-binding molecule as disclosed herein in concentrations of from about 10 μg/ml to about 100 mg/ml. In some embodiments, the concentrations of the D3-binding molecule (e.g., antibody or the antigen binding portion thereof) will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In some embodiments, the concentrations of the D3-binding molecule (e.g., antibody or the antigen binding portion thereof) will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

### Applications of the Disclosure

The antibodies, antibody compositions and methods of the present disclosure have numerous in vitro and in vivo utilities and uses including, for example, detection of D3 or enhancement of immune response. For example, these molecules can be administered to cells in culture, in vitro or ex vivo, or to human subjects, e.g., in vivo, to enhance immunity in a variety of situations. The immune response can be modulated, for example, augmented, stimulated or up-regulated.

For example, the subjects include human patients in need of enhancement of an immune response. The methods are particularly suitable for treating human patients having a disorder that can be treated by augmenting an immune response (e.g., a T-cell mediated immune response). In some embodiments, the methods are particularly suitable for treatment of cancer in vivo. To achieve antigen-specific enhancement of immunity, anti-D3 antibodies can be administered together with an antigen of interest or the antigen may already be present in the subject to be treated (e.g., a tumor-bearing or virus-bearing subject). When antibodies to D3 are administered together with another agent, the two can be administered in either order or simultaneously.

The present disclosure further provides methods for detecting the presence of human D3 antigen in a sample, or measuring the amount of human D3 antigen, comprising contacting the sample, and a control sample, for example, with a human monoclonal antibody, or an antigen binding portion thereof, which specifically binds to human D3, under conditions that allow for formation of a complex between the antibody or portion thereof and human D3. The formation of a complex is then detected, wherein a difference complex formation between the sample compared to the control sample is indicative of the presence of human D3 antigen in the sample. Moreover, anti-D3 antibodies of the disclosure can be used to purify human D3 via immunoaffinity purification.

### Treatment of Disorders Including Cancers

In some aspects, the present disclosure provides a method of treating a disorder or a disease in a mammal, which comprises administering to the subject (for example, a human) in need of treatment a therapeutically effective amount of an anti-D3 antibody or antigen-binding portion thereof as disclosed herein. In some aspects, the present disclosure provides an anti-D3 antibody or antigen-binding portion thereof as disclosed herein for use in treating a disease or disorder. In some aspects, provided herein is a use of an anti-D3 antibody or antigen-binding portion thereof as disclosed herein for the manufacture of a medicament for the treatment of a disease or disorder. The disorder or disease may be a cancer.

A variety of cancers where D3 is implicated, whether malignant or benign and whether primary or secondary, may be treated or prevented with a method provided by the disclosure. The cancers may include, but not limited to, lung (including various subtypes, e.g. small cell and non-small cell lung cancer), adrenal, liver, kidney, bladder, breast, gastric, ovarian, cervical, uterine, esophageal, colorectal, prostate pancreatic, thyroid, carcinomas, sarcomas, glioblastomas and various head and neck tumors. Exemplary cancers include, for example, small cell lung cancer, large cell neuroendocrine carcinoma, glioblastoma, Ewing's sarcoma, and cancers with neuroendocrine phenotype.

Anti-D3 antibodies as disclosed herein can be used for treating lung cancers such as bronchogenic carcinoma, non-small cell lung cancer, squamous cell carcinoma, small cell carcinoma, large cell carcinoma, and adenocarcinoma, e.g. lung adenocarcinoma. The lung cancers may be refractory, relapsed or resistant to a platinum based agent (e.g., carboplatin, cisplatin, oxaliplatin, topotecan) and/or a taxane (e.g., docetaxel, paclitaxel, larotaxel or cabazitaxel).

Cancers to be treated by anti-D3 antibodies as disclosed herein may also be large cell neuroendocrine carcinoma (LCNEC), medullary thyroid cancer, glioblastoma, neuroendocrine prostate cancer (NEPC), high-grade gastroenteropancreatic cancer (GEP) and malignant melanoma. Anti-D3 antibodies as disclosed herein may be used to treat neuroendocrine tumors (both NET and pNET) arising in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma).

As described above, anti-D3 antibodies are especially effective at treating lung cancer, including the following subtypes: small cell lung cancer and non-small cell lung cancer (e.g. squamous cell non-small cell lung cancer or squamous cell small cell lung cancer) and large cell neuroendocrine carcinoma.

### Stimulation of an Immune Response

In some aspects, the disclosure also provides a method of enhancing (for example, stimulating) an immune response in a subject comprising administering to the subject a D3-binding molecule, for example, an anti-D3 antibody or an antigen binding portion thereof, of the disclosure such that an immune response in the subject is enhanced. In some aspects, the present disclosure provides an anti-D3 antibody or antigen-binding portion thereof as disclosed herein for use in enhancing (for example, stimulating) an immune response in a subject. In some aspects, provided herein is a use of an anti-D3 antibody or antigen-binding portion thereof as disclosed herein for the manufacture of a medicament for enhancing (for example, stimulating) an immune response in a subject. For example, in some embodiments, the subject is a mammal. In some embodiments, the subject is a human.

The term “enhancing an immune response” or its grammatical variations, means stimulating, evoking, increasing, improving, or augmenting any response of a mammal's immune system. The immune response may be a cellular response (e.g. cell-mediated, such as cytotoxic T lymphocyte mediated) or a humoral response (e.g. antibody mediated response), and may be a primary or secondary immune response. Examples of enhancement of immune response include increased CD4⁺ helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example 1L-2 production or IFN-γ production), regression of tumors, survival of tumor bearing animals, antibody production, immune cell proliferation, expression of cell surface markers, and cytotoxicity. For example, methods of the disclosure are useful to enhance the immune response by a mammal when compared to the immune response by an untreated mammal or a mammal not treated using the methods as disclosed herein.

A D3-binding molecule may be used alone as a monotherapy, or may be used in combination with chemical therapies, radiotherapies, targeted therapies or cell immunotherapies etc.

Combined Use with Chemotherapies

A D3-binding molecule (e.g., an anti-D3 antibody) may be used in combination with chemotherapies, including, for example, an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in some embodiments as discussed above, such anti-cancer agents may comprise conjugates and may be associated with the disclosed anti-D3 antibodies prior to administration. For example, in some embodiments selected anti-cancer agents will be linked to the unpaired cysteines of the engineered antibodies to provide engineered conjugates (e.g., antibody-drug conjugates) as set forth herein. Accordingly, such engineered conjugates are expressly contemplated as being within the scope of the present disclosure. In some embodiments, the disclosed anti-cancer agents will be given in combination with anti-D3 conjugates comprising a different therapeutic agent as set forth above.

As used herein the term “cytotoxic agent” means a substance that is toxic to the cells and decreases or inhibits the function of cells and/or causes destruction of cells. In some embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with D3-binding molecules (e.g., anti-D3 antibodies) of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, OR), razoxane; rhizoxin; sizofiran; spirogermanium; tenuazonic acid; triaziquone; 2,2′,2 ″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Combined Use with Radiotherapies

The present disclosure also provides for the combination of a D3-binding molecule with a radiotherapy (e.g., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed D3-binding molecules may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### Diagnosis

The disclosure provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with an anti-D3 antibody as described herein and detecting presence or absence, or level of association, of the antibody to bound or free target molecules in the sample. In some embodiments, the anti-D3 antibody will comprise a detectable label or reporter molecule as described herein.

In some embodiments, the association of an anti-D3 antibody with particular cells in the sample can denote that the sample may contain tumorigenic cells, thereby indicating that the individual having cancer may be effectively treated with an anti-D3 antibody as described herein.

Samples can be analyzed by numerous assays, for example, radioimmunoassays, enzyme immunoassays (e.g. ELISA), competitive-binding assays, fluorescent immunoassays, immunoblot assays, Western Blot analysis and flow cytometry assays. Compatible in vivo theragnostic or diagnostic assays can comprise art recognized imaging or monitoring techniques, for example, magnetic resonance imaging, computerized tomography (e.g. CAT scan), positron tomography (e.g., PET scan), radiography, ultrasound, etc., as would be known by those skilled in the art.

### Pharmaceutical Packs and Kits

Pharmaceutical packs and kits comprising one or more containers, comprising one or more doses of a D3-binding molecule are also provided. In some embodiments, a unit dosage is provided wherein the unit dosage contains a predetermined amount of a composition comprising, for example, a D3-binding molecule, with or without one or more additional agents. In some embodiments, such a unit dosage is supplied in single-use prefilled syringe for injection. In some embodiments, the composition contained in the unit dosage may comprise saline, sucrose, or the like; a buffer, such as phosphate, or the like; and/or be formulated within a stable and effective pH range. Alternatively, in some embodiments, a composition may be provided as a lyophilized powder that may be reconstituted upon addition of an appropriate liquid, for example, sterile water or saline solution. In some embodiments, the composition comprises one or more substances that inhibit protein aggregation, including, but not limited to, sucrose and arginine. Any label on, or associated with, the container(s) indicates that the enclosed composition is used for treating the neoplastic disease condition of choice.

The present disclosure also provides kits for producing single-dose or multi-dose administration units of a D3-binding molecule and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed D3-binding molecules in a conjugated or unconjugated form. In some embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of a D3-binding molecule in a conjugated or unconjugated form and, optionally, one or more anti-cancer agents in the same or different containers. The kits may also contain other pharmaceutically acceptable formulations, either for diagnosis or combined therapy. For example, in addition to a D3-binding molecule of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

For example, the kits may have a single container that contains a D3-binding molecule, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, for example, a sterile aqueous or saline solution. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer a D3-binding molecule and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

Appended to the instant application is a sequence listing comprising a number of amino acid sequences. The following Tables A-F provide a summary of the included sequences. The exemplary antibodies are collectively referred to as “WBPT1156 antibodies” in the present disclosure.

TABLE A
Amino acid sequences of the CDR region

| VHH | CDR1 | CDR2 | CDR3 |
|---|---|---|---|
| WT1156-P3R2-1C2 | SEQ ID NO: 1 | SEQ ID NO: 2 | SEQ ID NO: 3 |
| WT1156-P3R2-1C2-z102 | GLTFSTATVG | AIPAYYSTYY | DDTPSPSRSPF |
| WT1156-P3R2-1C2-z109 | | ASSVKG | YKH |
| WT1156-P3R2-1C2-z109′ | | | |
| WT1156-P3R2-1C9 | SEQ ID NO: 4 | SEQ ID NO: 5 | SEQ ID NO: 6 |
| | GRTTSRYSMV | GNSAHDGRS | DTNPPYGPPW |
| | | AYADSVKG | STPSEYEY |
| WT1156-P3R2-1H6 | SEQ ID NO: 7 | SEQ ID NO: 8 | SEQ ID NO: 9 |
| WT1156-P3R2-1H6-z100 | GRTFRSYAMG | AISWIGGGTY | SSLLRHGHMF |
| | | YADSVKG | EESDY |
| WT1156-P8R2-1H1 | SEQ ID NO: 10 | SEQ ID NO: 11 | SEQ ID NO: 6 |
| | GRTASRYSMV | GNSAHDGRS | DTNPPYGPPW |
| | | AYTDSVKG | STPSEYEY |

TABLE B
Amino acid sequences of the VHH region and human IgG1 heavy chain Fc region

| | Amino acid | Amino acid sequence |
|---|---|---|
| | sequence of VHH region | of human IgG1 Fc region |
| WT1156-P3R2-1C2 | SEQ ID No: 12 | SEQ ID No: 19 |
| | EVQLVESGGGLVQTGDSLRLSCAASGL | DKTHTCPPCPAPELLGGP |
| | TFSTATVGWFRQAPGKERDLIAAIPAYY | SVFLFPPKPKDTLMISRTP |
| | STYYASSVKGRFTISRDNAKNTVYLQM | EVTCVVVDVSHEDPEVK |
| | NSLKPEDTGVYYCAADDTPSPSRSPFYK | FNWYVDGVEVHNAKTK |
| | HRGQGTQVTVSS | PREEQYNSTYRVVSVLTV |
| WT1156-P3R2-1C9 | SEQ ID No: 13 | LHQDWLNGKEYKCKVS |
| | QVQLVESGGGLVQAGGSLRLSCAASGR | NKALPAPIEKTISKAKGQ |
| | TTSRYSMVWFRQAPGQEREFVGGNSA | PREPQVYTLPPSREEMTK |
| | HDGRSAYADSVKGRFTFSRDNAKNTGY | NQVSLTCLVKGFYPSDIA |
| | LQMSSLRPDDTAVYYCAADTNPPYGPP | VEWESNGQPENNYKTTP |
| | WSTPSEYEYWGHGTQVTVSS | PVLDSDGSFFLYSKLTVD |
| WT1156-P3R2-1H6 | SEQ ID No: 14 | KSRWQQGNVFSCSVMHE |
| | QVQLVESGGGLVQAGGSLRLSCAASGR | ALHNHYTQKSLSLSPGK |
| | TFRSYAMGWFRQAPGKEREFVAAISWI | |
| | GGGTYYADSVKGRFTISGDNAKNTLYL | |
| | QMNSLKPEDTAVYYCAASSLLRHIGHM | |
| | FEESDYWGQGTQVTVSS | |
| WT1156-P8R2-1H1 | SEQ ID No: 15 | |
| | EVDLVESGGGLVQPGGSLRLSCAASGR | |
| | TASRYSMVWFRQAPGQEREFVGGNSA | |
| | HDGRSAYTDSVKGRFTFSRDNAKNTGY | |
| | LQMNSLRPDDTAVYYCAADTNPPYGPP | |
| | WSTPSEYEYWGHGTQVTVSS | |
| WT1156-P3R2-1C2- | SEQ ID No: 16 | |
| z102 | EVQLVESGGGLVQPGGSLRLSCAASGLT | |
| | FSTATVGWFRQAPGKGRELIAAIPAYYS | |
| | TYYASSVKGRFTISRDNAKNSVYLQMN | |
| | SLRAEDTAVYYCAADDTPSPSRSPFYK | |
| | HRGQGTMVTVSS | |
| WT1156-P3R2-1C2- | SEQ ID No: 17 | |
| z109 | EVQLVESGGGLVQPGGSLRLSCAASGLT | |
| | FSTATVGWFRQAPGKGRELVAAIPAYY | |
| | STYYASSVKGRFTISRDNAKNSLYLQM | |
| | NSLRPEDTAVYYCAADDTPSPSRSPFYK | |
| | HRGQGTMVTVSS | |
| WT1156-P3R2-1H6- | SEQ ID No: 18 | |
| z100 | QVQLVESGGGVVQPGGSLRLSCAASGR | |
| | TFRSYAMGWFRQAPGKEREFVAAISWI | |
| | GGGTYYADSVKGRFTISGDNSKNTLYL | |
| | QMNSLRAEDTAVYYCAASSLLRHGHM | |
| | FEESDYWGQGTMVTVSS | |

TABLE C
Amino acid sequences of Antibody Clones WT1156-P3R2-1C2, WT1156-P3R2-
1C2-z102, WT1156-P3R2-1C2-z109 and WT1156-P3R2-1C2-z109'

| | | Exemplary | IMGT | Kabat | Chothia | Contact | AbM |
|---|---|---|---|---|---|---|---|
| VHH | VHH | GLTFSTATVG | GLTFSTAT | TATVG | GLTFSTA | STATVG | GLTFSTATVG |
| CDR | CDR1 | (SEQ ID | (SEQ ID | (SEQ ID | (SEQ ID | (SEQ ID | (SEQ ID NO: 1) |
| Seq. | | NO: 1) | NO: 20) | NO: 23) | NO: 24) | NO: 27) | |
| | VHH | AIPAYYSTYY | IPAYYST | AIPAYYSTYYA | AYY | LIAAIPAYYST | AIPAYYSTY |
| | CDR2 | ASSVKG | (SEQ ID | SSVKG | (SEQ ID | Y | (SEQ ID NO: 30) |
| | | (SEQ ID | NO: 21) | (SEQ ID | NO: 25) | (SEQ ID | |
| | | NO: 2) | | NO: 2) | | NO: 28); or | |
| | | | | | | LVAAIPAYYS | |
| | | | | | | TY | |
| | | | | | | (SEQ ID | |
| | | | | | | NO: 56) | |
| | VHH | DDTPSPSRSPF | AADDTPSPSRS | DDTPSPSRSPFY | DTPSPSRSPF | AADDTPSPSR | DDTPSPSRSPF |
| | CDR3 | YKH | PFYKH | KH | YK | SPFYK | YKH |
| | | (SEQ ID | (SEQ ID | (SEQ ID | (SEQ ID | (SEQ ID | (SEQ ID NO: 3) |
| | | NO: 3) | NO: 22) | NO: 3) | NO: 26) | NO: 29) | |

VHH Sequence WT1156-P3R2-1C2:
EVQLVESGGGLVQTGDSLRLSCAASGLTFSTATVGWFRQAPGKERDLIAAIPAYYSTYYASSVKGRFTISRDNAKNTVYLQMN
SLKPEDTGVYYCAADDTPSPSRSPFYKHRGQGTQVTVSS (SEQ ID No: 12)
VHH Sequence WT1156-P3R2-1C2-z102
EVQLVESGGGLVQPGGSLRLSCAASGLTFSTATVGWFRQAPGKGRELIAAIPAYYSTYYASSVKGRFTISRDNAKNSVYLQMNS
LRAEDTAVYYCAADDTPSPSRSPFYKHRGQGTMVTVSS (SEQ ID No: 16)
VHH Sequence WT1156-P3R2-1C2-z109:
EVQLVESGGGLVQPGGSLRLSCAASGLTFSTATVGWFRQAPGKGRELVAAIPAYYSTYYASSVKGRFTISRDNAKNSLYLQMN
SLRPEDTAVYYCAADDTPSPSRSPFYKHRGQGTMVTVSS (SEQ ID No: 17)
VHH Sequence WT1156-P3R2-1C2-z109':
EVQLVESGGGLVQPGGSLRLSCAASGLTFSTATVGWFRQAPGKGRELIAAIPAYYSTYYASSVKGRFTISRDNAKNSLYLQMNS
LRPEDTAVYYCAADDTPSPSRSPFYKHRGQGTMVTVSS (SEQ ID No: 55)

TABLE D
Amino acid sequences of Antibody Clone WT1156-P3R2-1C9

| | | Exemplary | IMGT | Kabat | Chothia | Contact | AbM |
|---|---|---|---|---|---|---|---|
| VHH | VHH | GRTTSRYSMV | GRTTSRYS | RYSMV | GRTTSRY | SRYSMV | GRTTSRYSMV |
| CDR | CDR1 | (SEQ ID | (SEQ ID | (SEQ ID | (SEQ ID | (SEQ ID | (SEQ ID NO: 4) |
| Seq. | | NO: 4) | NO: 31) | NO: 34) | NO: 35) | NO: 38) | |
| | VHH | GNSAHDGRSA | NSAHDGRS | GNSAHDGRSA | AHDG | FVGGNSAHDGR | GNSAHDGRSA |
| | CDR2 | YADSVKG | (SEQ ID | YADSVKG | (SEQ ID | SA | (SEQ ID NO: 41) |
| | | (SEQ ID | NO: 32) | (SEQ ID | NO: 36) | (SEQ ID | |
| | | NO: 5) | | NO: 5) | | NO: 39) | |
| | VHH | DTNPPYGPPW | AADTNPPYGP | DTNPPYGPPW | TNPPYGPPWS | AADTNPPYGPP | DTNPPYGPPW |
| | CDR3 | STPSEYEY | PWSTPSEYEY | STPSEYEY | TPSEYE | WSTPSEYE | STPSEYEY |
| | | (SEQ ID | (SEQ ID | (SEQ ID | (SEQ ID | (SEQ ID | (SEQ ID NO: 6) |
| | | NO: 6) | NO: 33) | NO: 6) | NO: 37) | NO: 40) | |

VHH Sequence WT1156-P3R2-1C9:
QVQLVESGGGLVQAGGSLRLSCAASGRTTSRYSMVWFRQAPGQEREFVGGNSAHDGRSAYADSVKGRFTFSRDNAKNTGYL
QMSSLRPDDTAVYYCAADTNPPYGPPWSTPSEYEYWGHGTQVTVSS (SEQ ID No: 13)

TABLE E
Amino acid sequences of Antibody Clones WT1156-P3R2-1H6, WT1156-P3R2-
1H6-z100

| | | Exemplary | IMGT | Kabat | Chothia | Contact | AbM |
|---|---|---|---|---|---|---|---|
| VHH | VHH | GRTFRSYAMG | GRTFRSYA | SYAMG | GRTFRSY | RSYAMG | GRTFRSYAMG |
| CDR | CDR1 | (SEQ ID | (SEQ ID | (SEQ ID | (SEQ ID | (SEQ ID NO: 49) | (SEQ ID NO: 7) |
| Seq. | | NO: 7) | NO: 42) | NO: 45) | NO: 46) | | |
| | VHH | AISWIGGGTYY | ISWIGGGT | AISWIGGGTY | WIGG | FVAAISWIGGG | AISWIGGGTY |
| | CDR2 | ADSVKG | (SEQ ID | YADSVKG | (SEQ ID | TY | (SEQ ID NO: 52) |
| | | (SEQ ID | NO: 43) | (SEQ ID | NO: 47) | (SEQ ID NO: 50) | |
| | | NO: 8) | | NO: 8) | | | |
| | VHH | SSLLRHGHMF | AASSLLRHGH | SSLLRHGHM | SLLRHGHMFE | AASSLLRHGH | SSLLRHGHMFE |
| | CDR3 | EESDY | MFEESDY | FEESDY | ESD | MFEESD | ESDY |
| | | (SEQ ID | (SEQ ID | (SEQ ID | (SEQ ID | (SEQ ID NO: 51) | (SEQ ID NO: 9) |
| | | NO: 9) | NO: 44) | NO: 9) | NO: 48) | | |

VHH Sequence WT1156-P3R2-1H6:
QVQLVESGGGLVQAGGSLRLSCAASGRTFRSYAMGWFRQAPGKEREFVAAISWIGGGTYYADSVKGRFTISGDNAKNTLYLQ
MNSLKPEDTAVYYCAASSLLRHGHMFEESDYWGQGTQVTVSS (SEQ ID No: 14)
VHH Sequence WT1156-P3R2-1H6-z100:
QVQLVESGGGVVQPGGSLRLSCAASGRTFRSYAMGWFRQAPGKEREFVAAISWIGGGTYYADSVKGRFTISGDNSKNTLYLQ
MNSLRAEDTAVYYCAASSLLRHGHMFEESDYWGQGTMVTVSS (SEQ ID No: 18)

TABLE F
Amino acid sequences of Antibody Clone WT1156-P8R2-1H1

| | | Exemplary | IMGT | Kabat | Chothia | Contact | AbM |
|---|---|---|---|---|---|---|---|
| VHH | VHH | GRTASRYSMV | GRTASRYS | RYSMV | GRTASRY | SRYSMV | GRTASRYSMV |
| CDR | CDR1 | (SEQ ID | (SEQ ID | (SEQ ID | (SEQ ID | (SEQ ID NO: 38) | (SEQ ID NO: 10) |
| Seq. | | NO: 10) | NO: 53) | NO: 34) | NO: 54) | | |
| | VHH | GNSAHDGRSA | NSAHDGRS | GNSAHDGRSA | AHDG | FVGGNSAHDG | GNSAHDGRSA |
| | CDR2 | YTDSVKG | (SEQ ID | YTDSVKG | (SEQ ID | RSA | (SEQ ID NO: 41) |
| | | (SEQ ID | NO: 32) | (SEQ ID | NO: 36) | (SEQ ID NO: 39) | |
| | | NO: 11) | | NO: 11) | | | |
| | VHH | DTNPPYGPPW | AADTNPPYGP | DTNPPYGPPW | TNPPYGPPWST | AADTNPPYGP | DTNPPYGPPW |
| | CDR3 | STPSEYEY | PWSTPSEYEY | STPSEYEY | PSEYE | PWSTPSEYE | STPSEYEY |
| | | (SEQ ID | (SEQ ID | (SEQ ID | (SEQ ID | (SEQ ID NO: 40) | (SEQ ID NO: 6) |
| | | NO: 6) | NO: 33) | NO: 6) | NO: 37) | | |

VHH Sequence WT1156-P8R2-1H1:
EVDLVESGGGLVQPGGSLRLSCAASGRTASRYSMVWFRQAPGQEREFVGGNSAHDGRSAYTDSVKGRFTFSRDNAKNTGYLQ
MNSLRPDDTAVYYCAADTNPPYGPPWSTPSEYEYWGHGTQVTVSS (SEQ ID No: 15)

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Antigens, Benchmark Antibodies and Cell Lines

### 1.1 Antigen Generation

DNA sequences encoding the extracellular domain (LCD) sequence of cynomolgus D3 (Uniprot No. A0A2K5WSR4) and mouse D3 (Uniprot No. 088516), were synthesized in Sangon Biotech (Shanghai, China), and then subcloned into modified pcDNA3.3 expression vectors with MBP tag in N-terminal and AVI-His tag or human Fc tag in C-terminal. Human D3 (Uniprot No. Q9NYJ7) was purchased from AcroBiosystems (Cat. DL3-H52H4).

DNA sequences (as disclosed in WO 2017/021349) encoding the truncated isoforms of human D3 were synthesized in Sangon Biotech (Shanghai, China), and then subcloned into modified pcDNA3.3 expression vectors with MBP tag and AVI-His tag in C-terminal.

Expi293 cells (Invitrogen-A14527) were transfected with the purified expression vectors. Cells were cultured for 5 days and supernatant was collected for protein purification using Ni-NTA column (GE Healthcare, Cat. 175248) or Protein A column (GE Healthcare, Cat. 175438). The obtained mouse D3, cynomolgus D3 and truncated human D3 were analyzed by SDS-PAGE and SEC, and then stored at −80° C.

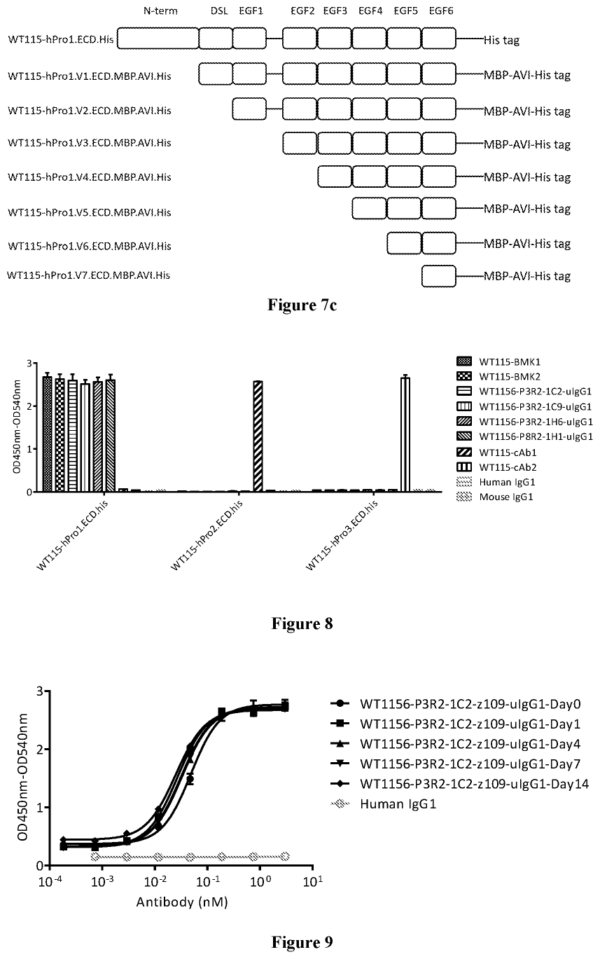

Human D3 (ACRO DL3-H52H4) was named as WT115-hPro1.ECD.His. The obtained mouse D3 was named as WT115-MBP-mPro1.ECD.hFc. Human D3 protein is characterized by a Delta/Serrate/LAG-2 (DSL) domain, six epidermal growth factor (EGF)-like repeats (EGF domain), and a transmembrane domain. Truncated isoforms of human D3 were named WT115-hPro1.V1.ECD.MBP.AVI.His (DSL domain+EGF1-6 domain+membrane proximal), WT115-hPro1.V2.ECD.MBP.AVI.His (EGF1-6 domain+membrane proximal), WT115-hPro1.V3.ECD.MBP.AVI.His (EGF2-6 domain+membrane proximal), WT115-hPro1.V4.ECD.MBP.AVI.His (EGF3-6 domain+membrane proximal), WT115-hPro1.V5.ECD.MBP.AVI.His (EGF4-6 domain+membrane proximal), WT115-hPro1.V6.ECD.MBP.AVI.His (EGF5-6 domain+membrane proximal), WT115-hPro1.V7.ECD.MBP.AVI.His (EGF6 domain+membrane proximal). The diagrams of the truncated protein are shown in FIG. 7c.

### 1.2 Construction of Expression Vector of BMK Antibodies

Two anti-D3 antibodies were used as benchmark antibodies and referred to as WT115-BMK1 and WT115-BMK2 herein. DNA sequences encoding the variable region of WT115-BMK1 (SEQ ID NO: 212 and SEQ ID NO: 213 in US 2019/0046656) and WT115-BMK2 (SEQ ID NO: 37 and SEQ ID NO: 38 in WO 2017/021349) were synthesized in Sangon Biotech (Shanghai, China), and then subcloned into modified pcDNA3.3 expression vectors encoding an Fc region of human IgG1.

The plasmid containing VH and VL gene were co-transfected into Expi293 cells. Cells were cultured for 5 days and supernatant was collected for protein purification using Protein A column (GE Healthcare, 175438). The obtained antibodies were analyzed by SDS-PAGE and SEC, and then stored at −80° C.

### 1.3 Establishment of Stable Cell Lines/Cell Pool

Using Lipofectamine 2000, 293F cells were transfected with the expression vector containing gene encoding full length human D3 (UniProt, Q9NYJ7-1). Flpin293 cells were transfected with the expression vector containing gene encoding full length cynomolgus D3. Cells were cultured in medium containing proper selection marker. Human D3 high expression stable cell line (WT115-293F.hPro1.2E5) were selected after limited dilution, and cynomolgus D3 high expression stable cell pool (WT115.Flpin293.cPro1.pool) with proper selection antibiotics.

### 1.4 Antibodies Biotinylation

For NHS-PEO4-Biotinylation, 1-10 mg/mL antibodies (IgG) were incubated with 20-fold molar excess of NHS-PEO4-Biotin reagent at 25° C. for 75 minutes in metal bath or on ice for two hours. Excess biotin was then removed using a desalting spin column, and the purified protein sample was collected from the flow-through solution. The level of biotin incorporation in the protein was determined by HABA assay: dilute the biotinylated sample 10-fold with HABA/Avidin solution and measure the absorbance of the mixed solution at A500. The moles of biotin per mole of protein were calculated based on the A500 value.

### Example 2

### Production of VHH-Comprising WBPT1156 Antibodies

### 2.1 Generation of Anti-D3 VHHs

Anti-D3 VHHs were generated by immunization of Camelidae animals and phage display technology. Briefly, Alpacas (Vicugna pacos) were subcutaneously immunized with hFc tagged human D3 ECD protein (ACRO, DL3-H5255). After immunization, peripheral blood was collected for construction of phage library displaying VHH fragments. After bio-panning with corresponding target ECD proteins, the positive VHH clones binding to D3 were selected.

### 2.2 VHH Sequencing

The positive E. co/i clones selected by target specific binding ELISA and FACS with E. coli supernatants were sent to Biosune (Shanghai, China) for nucleotide sequencing of VHH gene. The sequencing results were analyzed using CLC Main Workbench (Qiagen, Hilden, Germany). The sequences of 4 unique positive VHH clones were WT1156-P3R2-1C2, WT1156-P3R2-1C9, WT1156-P3R2-1H6 and WT1156-P8R2-1H1 shown in Table A and B.

### 2.3 Generation of Human Fc Fusion Antibodies Comprising the VHHs

The 4 unique positive VHH clones were converted to VHH-Fc (hIgG1) fusion antibodies. Briefly, the VHH genes were PCR amplified from the pET-bac vectors using VHH-specific cloning primers containing appropriate restriction sites then cloned by fusion into a modified human hIgG1 expression pcDNA3.3 vector to create corresponding clones of VHH-Fc (hIgG1) chimeric antibody. 293F or Expi293 cells were transiently transfected with the vectors for antibody expression. The cell culture supernatants containing antibodies were harvested and purified using Protein A chromatography. The generated antibodies were named as “WT1156-P3R2-1C2-uIgG1”, “WT1156-P3R2-1C9-uIgG1”, “WT1156-P3R2-1H6-uIgG1” and “WT1156-P8R2-1H1-uIgG1”, respectively. The obtained antibodies were analyzed by SDS-PAGE and HPLC-SEC, and then stored at −80° C.

### 2.4 Humanization

VHH humanization was done by “Best Fit” approach. Briefly, amino acid sequences of VHH framework regions were blasted against human germline V-gene database, and humanized VHH sequences were generated by replacing human CDR sequences in the top hit with VHH CDR sequences using Kabat CDR definition. Then key residues in framework which play an important role in antibody affinity or developability were back mutated to parental residues alone or in combination. The variants were codon optimized for mammalian expression and then synthesized by GENEWIZ (SuZhou, China). The designed VHH variants and parental VHH proteins were cloned into human IgG1 expression vectors to generate human IgG1 constructs. Antibodies were produced in HEK293 cells and purified using Protein A chromatography. The variants with desired affinity were finally selected as the humanized leads.

The sequences of three unique humanized D3 antibodies, WT1156-P3R2-1C2-z102-uIgG1, WT1156-P3R2-1C2-z109-uIgG1 and WT1156-P3R2-1H6-z100-uIgG1 are also shown in Table A and B.

### Example 3

### Characterization of D3-Binding Antibodies

### 3.1 Human D3 Binding by ELISA

Plates were pre-coated with 1 μg/mL, 100 μL per well of WT115-hPro1.ECD.His at 4° C. overnight. The antigen was diluted in coating buffer (0.02 M Na₂CO₃ and 0.18 M NaHCO₃, pH9.2) from stock solution. Next day, the plates were washed using 1×PBST (PBS containing 0.05% tween-20) for one time, and blocking was done by adding 200 μL of 1×PBS/2% BSA. Antibodies were serially diluted (5-fold serially diluted from 20 nM to 0.00128 nM) in blocking buffer. After 1-hour blocking, the plates were washed using 1×PBST for 3 times, and then the antibodies were added to the plates and incubated at ambient temperature for 1 hour. Binding of antibodies to the immobilized human D3 was detected by HRP-labeled secondary antibody (Bethyl, A80-304P), which was diluted in 1×PBS/2% BSA at a concentration of 1:10000. After incubation, the plates were washed using 1×PBST for 6 times. The color was developed by dispensing 100 μL of TMB substrate, and then reaction was stopped by adding 100 μL of 2M HCl. Absorbance was read at 450 nm and 540 nm using a microplate spectrophotometer. Anti-human D3 antibodies WT115-BMK1 and WT115-BMK2 were used as positive controls. Human IgG1 isotype antibody was used as a negative control. All samples were tested in duplicate.

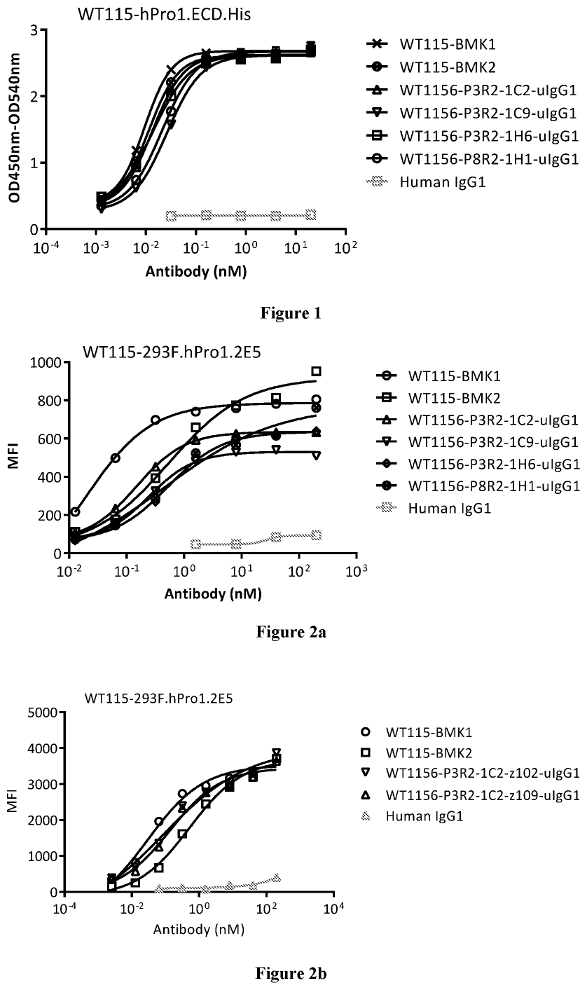

As shown in FIG. 1 and Table 1, WT1156-P3R2-1C2-uIgG1, WT1156-P3R2-1C9-uIgG1, WT1156-P3R2-1H6-uIgG1 and WT1156-P8R2-1H1-uIgG1 can strongly bind to immobilized human D3, comparable to WT1115-BMK1 and WT115-BMK2. EC50 of WT1156 antibodies ranges from 0.013 nM to 0.026 nM. EC50 of WT115-BMK1 and WT115-BMK2 is 0.0094 nM and 0.011 nM, respectively.

TABLE 1
Characterization summary of WBPT1156 antibodies.

| | Human D3 | Human D3 | Cyno D3 | | |
|---|---|---|---|---|---|
| | binding ELISA | binding FACS | binding FACS | Internalization | Binding |
| Antibody name | (EC50, nM) | (EC50, nM) | EC50, nM) | EC50, nM) | domain |
| WT1156-P3R2- | 0.013 | 0.15 | 0.14 | 0.65 | EGF1-EGF2 |
| 1C2-uIgG1 | | | | | |
| WT1156-P3R2- | 0.014 | 0.52 | 0.25 | 0.95 | N-Term |
| 1H6-uIgG1 | | | | | |
| WT1156-P8R2- | 0.021 | 0.45 | 0.21 | 0.47 | N-DSL-EGF1 |
| 1H1-uIgG1 | | | | | |
| WT1156-P3R2- | 0.026 | 0.25 | 0.07 | 0.46 | N-Term |
| 1C9-uIgG1 | | | | | |
| WT115-BMK1 | 0.0094 | 0.019 | na | 0.19 | DSL |
| WT115-BMK2 | 0.011 | 0.54 | na | 0.58 | EGF3 |

na: The EC50 value was not fitted.

### 3.2 Human D3 Binding by FACS

WT115-293F.hPro1.2E5 cells (1×10⁵ cells/well) were incubated with various concentrations of antibodies (5-fold serially diluted from 200 nM to 0.0128 nM) at 4° C. for 1 hour. After washing with 1×PBS/1% BSA, a secondary antibody, R-PE-labeled goat anti-human IgG (1:150, Jackson ImmunoResearch, 109-115-098), was added and incubated with cells at 4° C. in dark for 1 hour. Anti-human D3 antibodies WT115-BMK1 and WT115-BMK2 were used as positive controls. Human IgG1 isotype antibody was used as a negative control. The cells were washed and resuspended in 4% paraformaldehyde. MFI of the cells was measured by a flow cytometer and analyzed by FlowJo.

As shown in FIG. 2a and Table 1, WT1156-P3R2-1C2-uIgG1, WT1156-P3R2-1C9-uIgG1, WT1156-P3R2-1H6-uIgG1 and WT1156-P8R2-1H1-uIgG1 can bind to human D3 expressing cell, comparable to WT115-BMK2. EC50 of WT1156 antibodies ranges from 0.15 nM to 0.52 nM. EC50 of WT115-BMK1 and WT115-BMK2 is 0.019 nM and 0.54 nM, respectively.

As shown in FIG. 2b, WT1156-P3R2-1C2-z102-uIgG1 and WT1156-P3R2-1C2-z109-uIgG1 can strongly bind to human D3 expressing cell with EC50 of 0.088 nM and 0.14 nM, respectively. EC50 of WT115-BMK1 and WT115-BMK2 is 0.03 nM and 0.52 nM, respectively. The data of WT1156-P3R2-1C2-z109-uIgG and two BMK antibodies are also summarized in Table 2.

TABLE 2
Characterization summary of WT1156-P3R2-1C2-z109-uIgG1.

| | Human D3 | Cyno D3 | Mouse D3 | |
|---|---|---|---|---|
| | binding FACS | Binding FACS | Binding ELISA | Internalization |
| Antibody name | (EC50, nM) | (EC50, nM) | (EC50, nM) | (EC50, nM) |
| WT1156-P3R2-1C2- | 0.14 | 0.096 | 0.0075 | 12.2 |
| z109-uIgG1 | | | | |
| WT115-BMK1 | 0.03 | 0.019 | 0.0039 | 3.47 |
| WT115-BMK2 | 0.52 | 0.20 | 0.014 | 14.4 |

### 3.3 Cynomolgus Monkey D3 Binding by FACS

WT115-Flpin293.cPro1.pool cells (1×10⁵ cells/well) were incubated with various concentrations of antibodies (4-fold serially diluted from 10 nM to 0.00061 nM) at 4° C. for 1 hour. After washing with 1×PBS/1% BSA, a secondary antibody, Alexa Fluor 647-labeled goat anti-human IgG (1:150, Jackson ImmunoResearch, 109-605-098), as added and incubated with cells at 4° C. in dark for 1 hour. Anti-human D3 antibodies WT115-BMIK1 and WT115-BMK2 were used as positive controls. Human IgG1 isotype antibody was used as a negative control. The cells were washed with 1×PBS/1% BSA and resuspended in 4% paraformaldehyde, and incubated with cells at 4° C. in dark for 0.5 hour. Then change the buffer with 1×PBS/1% BSA and filter the cells. MFI of the cells was measured by a flow cytometer and analyzed by FlowJo.

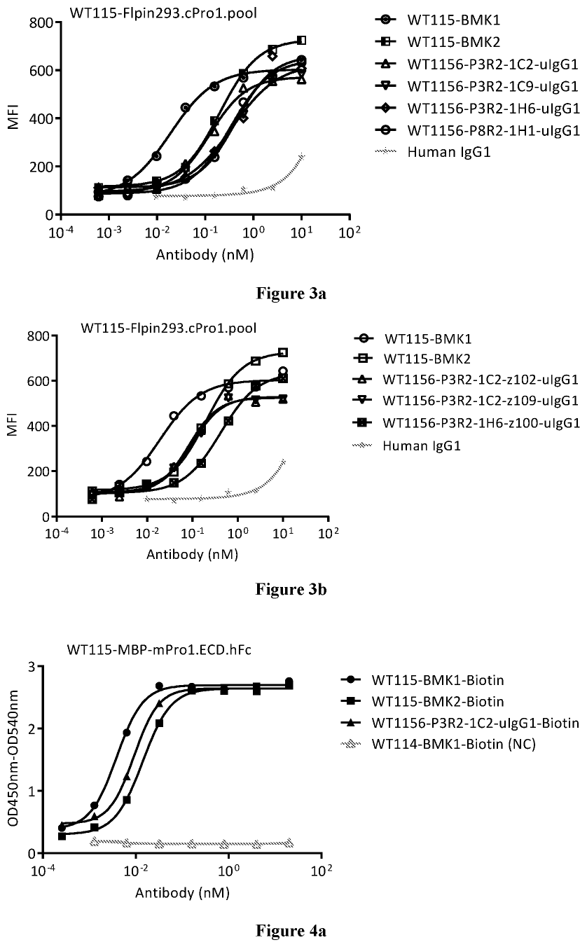

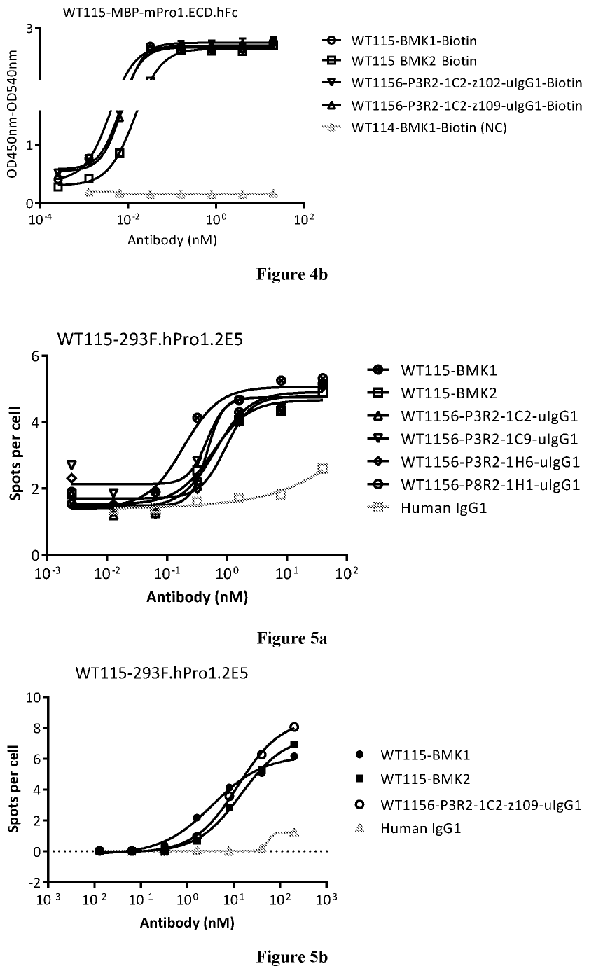

As shown in FIG. 3a and Table 1, WT1156-P3R2-1C2-uIgG1, WT1156-P3R2-1C9-uIgG1, WT1156-P3R2-1H6-uIgG1 and WT1156-P8R2-1H1-uIgG1 can bind to cynomolgus monkey D3 expressing cell, comparable to WT115-BMK1 and WT115-BMK2. EC50 of WT1156 antibodies ranges from 0.12 nM to 0.44 nM. EC50 of WT115-BMK1 and WT115-BMK2 is 0.019 nM and 0.20 nM, respectively.

As shown in FIG. 3b, WT1156-P3R2-1C2-z102-uIgG1, WT1156-P3R2-1C2-z109-uIgG1 and WT1156-P3R2-1H6-z100-uIgG1 can strongly bind to cynomolgus monkey D3 expressing cell with EC50 of 0.083 nM, 0.096 nM and 0.42 nM, respectively. EC50 of WT115-BMK1 and WT115-BMK2 is 0.019 nM and 0.20 nM, respectively. The data of WT1156-P3R2-1C2-z109-uIgG and two BMK are also summarized in Table 2.

### 3.4 Mouse D3 Binding by ELISA

Plates were pre-coated with 1 μg/mL, 100 μL per well of WT115-MBP-mPro1.ECD.hFc at 4° C. overnight. The antigen was diluted in coating buffer from stock solution. Next day, the plates were washed using 1×PBST for one time, and blocking was done by adding 200 μL of 1×PBS/2% BSA. Antibodies were serially diluted (5-fold serially diluted from 20 nM to 0.000256 nM) in blocking buffer. After 1-hour blocking, the plates were washed using 1×PBST for 3 times, and then the antibody were added to the plates and incubated at ambient temperature for 1 hour. Anti-human D3 antibodies WT115-BMK1-Biotin and WT 115-BMK2-Biotin were used as positive controls. WT114-BMK1-Biotin antibody was used as a negative control. Binding of antibodies to the immobilized mouse D3 was detected by HRP-labeled secondary antibody (Invitrogen, SNN1004), which was diluted in 1×PBS/2% BSA at a concentration of 1:30000. After incubation, the plates were washed using 1×PBST for 6 times. The color was developed by dispensing 100 μL of TMB substrate, and then reaction was stopped by adding 100 μL of 2M HCl. Absorbance was read at 450 nm and 540 nm using a microplate spectrophotometer. All samples were tested in duplicate.

As shown in FIG. 4a, WT1156-P3R2-1C2-uIgG1 can strongly bind to mouse D3, comparable to WT115-BMK1. EC50 of WT1156-P3R2-1C2-uIgG1 is 0.0092 nM. EC50 of WT115-BMK1 and WT115-BMK2 is 0.0039 nM and 0.014 nM, respectively.

As shown in FIG. 4b, WT1156-P3R2-1C2-z102-uIgG1 and WT1156-P3R2-1C2-z109-uIgG1 can strongly bind to mouse D3 protein with EC50 of 0.0067 nM and 0.0075 nM, respectively. EC50 of WT115-BMK1 and WT115-BMK2 is 0.0039 nM and 0.014 nM, respectively. The data of WT1156-P3R2-1C2-z109-uIgG and two BMK are also summarized in Table 2.

### 3.5 Internalization

WT115-293F.hPro1.2E5 cells (4×10⁴ cells/well) were plated in 96-well plate and the medium was removed from the plate after centrifuge. Prepared 1× final maximum concentration of primary antibodies (5-fold serially diluted from 40 nM to 0.00256 nM, or 5-fold serially diluted from 200 nM to 0.0128 nM) and pHrodo (amine reactive, Thermo Fisher, P36011) labeled second antibodies (Affinipure F(ab′)2 fragment goat anti-human IgG, Jackson ImmunoResearch, 109-006-098, Ratio=1:1 molecule) dilutions were added into plates with cell culture medium and incubate at 37° C. for 5 hours. Anti-human D3 antibodies WT115-BMK1 and WT115-BMK2 were used as positive controls. Human IgG1 isotype antibody was used as a negative control. After incubation, cells were stained with reagent (cell nucleus-Hoechst33342, 1000 ng/ml; cytoplasm-Calcein AM, 1:2000 dilution in DPBS) and incubate the plate at 37° C. for 15 mins. Finally, cells were photographed with Operatta CLS and the antibody endocytosis was analyzed by parameter of “Spots per cell”.

As shown in FIG. 5a and Table 1, WT1156-P3R2-1C2-uIgG1, WT1156-P3R2-1C9-uIgG1, WT1156-P3R2-1H6-uIgG1 and WT1156-P8R2-1H1-uIgG1 showed dose-dependent internalization potency in human D3 expressing cells, comparable to WT115-BMK1 and WT115-BMK2. EC50 of WT1156 antibodies ranges from 0.46 nM to 0.95 nM. EC50 of WT115-BMK1 and WT115-BMK2 is 0.19 nM and 0.58 nM, respectively.

As shown in FIG. 5b and Table 2, WT1156-P3R2-1C2-z109-uIgG1 showed dose-dependent internalization potency in human D3 expressing cells with EC50 of 12.2 nM. EC50 of WT115-BMK1 and WT115-BMK2 is 3.47 nM and 14.4 nM, respectively.

### 3.6 Kinetic Binding Affinity of Anti-D3 Antibodies

The binding affinity of anti-D3 antibodies to human D3 ECD protein was detected by SPR assay using Biacore T200. Each antibody tested was captured on an anti-human IgG Fc antibody immobilized CM5 sensor chip (GE). WT115-hPro1.ECD.His at different concentrations were injected over the sensor chip at a flow rate of 30 μl/min for an association phase of 180 s, followed by 3600 s dissociation. The chip was regenerated by 10 mM glycine (pH 1.5) after each binding cycle.

As shown in Table 3, the experimental data to human D3 was fitted by steady state affinity model. The experimental data of WT-115-BMK1 to human D3 was fitted by heterogeneous ligand model. The other experimental data was fitted by 1:1 model using Langmiur analysis. The sensorgrams of blank surface and buffer channel were subtracted from the test sensorgrams. Molecular weight of 34 KDa was used to calculate the molar concentration of analyte. The affinities of the tested antibodies to human D3 are shown in Table 3.

TABLE 3
Binding kinetics of WBPT1156 antibodies.

| Antibody name | kₐ (1/Ms) | k_{d} (1/s) | KD (M) |
|---|---|---|---|
| WT1156-P3R2-1C2-uIgG1 | 1.92E+05 | 1.28E−05 | 6.68E−11 |
| WT1156-P3R2-1H6-uIgG1 | 1.08E+05 | <1.00E−05 | <9.26E−11 |
| WT1156-P8R2-1H1-uIgG1 | 2.00E+05 | 5.14E−05 | 2.57E−10 |
| WT1156-P3R2-1C9-uIgG1 | 1.80E+05 | 2.38E−04 | 1.32E−09 |
| WT1156-P3R2-1C2-z102-uIgG1 | 1.83E+05 | 1.78E−05 | 9.77E−11 |
| WT1156-P3R2-1C2-z109-uIgG1 | 1.85E+05 | 3.06E−05 | 1.65E−10 |
| WT1156-P3R2-1H6-z100-uIgG1 | 8.02E+04 | 1.74E−05 | 2.17E−10 |
| WT115-BMK1 | 2.10E+06 | 2.65E−03 | 1.26E−09 |
| (heterogeneous ligand) | 1.73E+06 | 1.50E−04 | 8.67E−11 |
| WT115-BMK2 | 4.04E+05 | 3.98E−05 | 9.87E−11 |

### 3.7 Epitope Binning by Competition ELISA

Plates were pre-coated with 1 μg/mL, 100 μL per well of WT115-hPro1.ECD.His at 4° C. overnight. The antigen was diluted in coating buffer (0.02 M Na2CO3 and 0.18 M NaHCO₃, pH9.2) from stock solution. Next day, the plates were washed using 1×PBST for one time, and blocking was done by using 200 μL of 1×PBS/2% BSA. VHH antibodies were serially diluted (5-fold serially diluted from 10 nM to 0.00013 nM) in blocking buffer and pre-mixed with constant concentration of whole IgG antibodies (0.02 nM). After 1-hour blocking, the plates were washed using 1×PBST for 3 times, and then VHH antibody/whole IgG antibody mixture were added to the plates and incubated at ambient temperature for 1 hour. Binding of whole IgG antibodies to the immobilized human D3 was detected by HRP-labeled secondary antibody (Bethyl, A80-304P), which was diluted in 1×PBS/2% BSA at a concentration of 1:10000. After incubation, the plates were washed using 1×PBST for 6 times. The color was developed by dispensing 100 μL of TMB substrate, and then reaction stopped by adding 100 μL of 2M HCl. Absorbance was read at 450 nm and 540 nm using a microplate spectrophotometer. All samples were tested in duplicate.

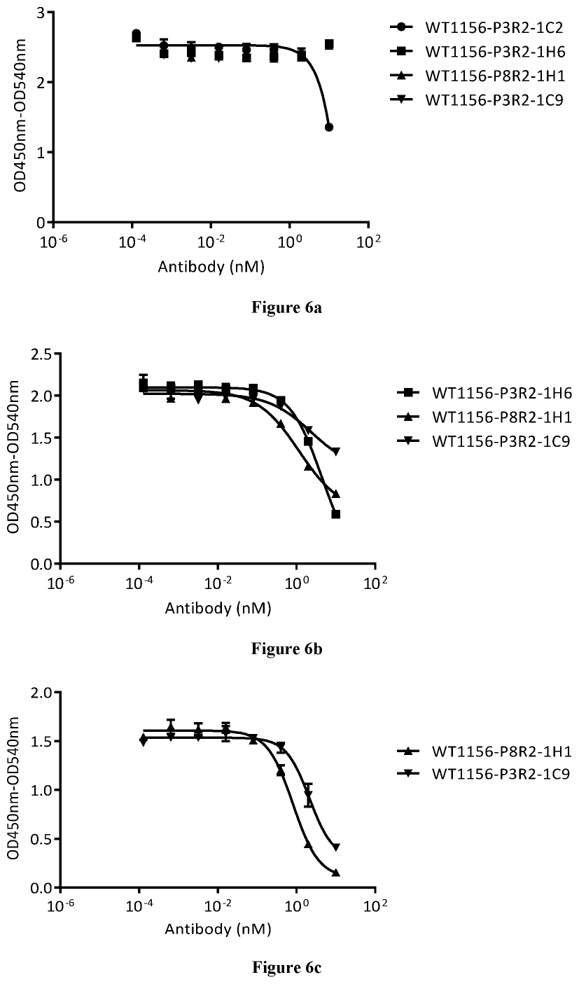

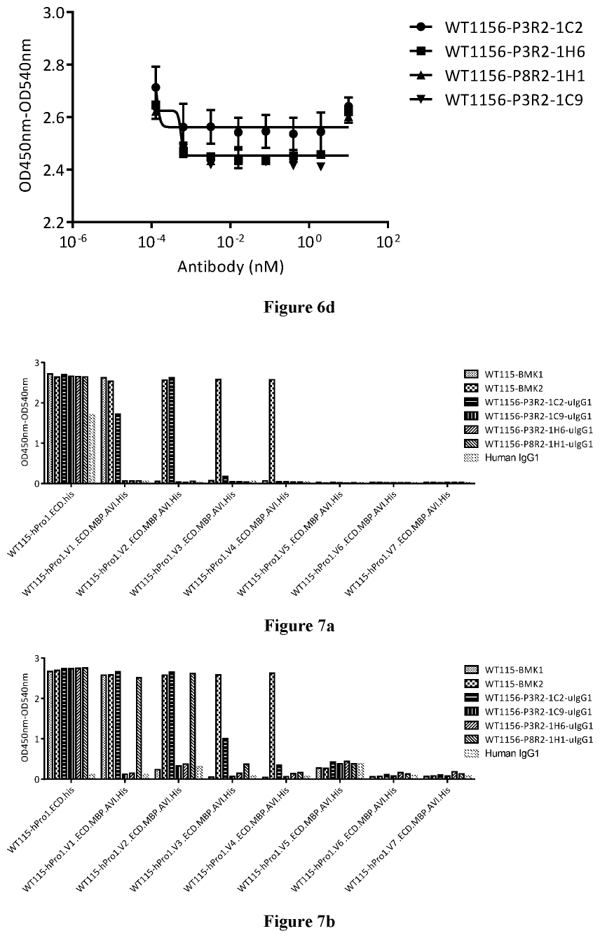

As shown in FIG. 6a, WT1156-P3R2-1C2.uIgG1 does not compete with the other 3 WBPT1156 antibodies for binding to human D3-ECD protein, tested by ELISA. And as shown in FIG. 6b, WT1156-P3R2-1H6-uIgG1 can compete with WT1156-P8R2-1H1-uIgG1 and WT1156-P3R2-1C9-uIgG1 for binding to human D3-ECD protein. FIG. 6c shows that WT1156-P8R2-1H1-uIgG1 can compete with WT1156-P3R2-1C9-uIgG1. As shown in FIG. 6d, WBPT1156 4 antibodies do not compete with WT115-BMK1.

3.8 Binding by ELISA with Truncated D3 Proteins

The binding epitopes of WT1156 antibodies were tested by ELISA with truncated D3 proteins, as described in 1.1 and FIG. 7c. The ELISA tests were performed by pre-coating plates with either antibodies or antigens. The results are shown in FIGS. 7a and 7b, respectively.

For ELISA with pre-coated antibodies, the ELISA Plates were pre-coated with 2 μg/mL, 100 μL per well of antibodies at 4° C. overnight. The antibody was diluted in coating buffer (0.02 M Na2CO3 and 0.18 M NaHCO₃, pH9.2) from stock solution. Next day, the plates were washed using 1×PBST for one time, and blocked with 200 μL of 1×PBS/2% BSA. Then the constant concentration of full-length DLLs ECD protein (WT115-hPro1.ECD.His)(3 μg/mL) or truncated D3 proteins (3 μg/mL or 6 μg/mL) diluted in blocking buffer were added. After 1-hour blocking, the plates were washed using 1×PBST for 3 times, and then antigen was added to the plates and incubated at ambient temperature for 1 hour. Binding of antigen to the immobilized WBPT1156 antibodies were detected by HRP-labeled secondary antibody (GenScript, A00612), which was diluted in 1×PBS/2% BSA at a concentration of 1:2000. After incubation, the plates were washed using 1×PBST for 6 times. The color was developed by dispensing 100 μL of TMB substrate. The color reaction was stopped by 2M HCl and the absorbance was read at 450 nm and 540 nm using a microplate spectrophotometer. All samples were tested in duplicate. The results are shown in FIG. 7a.

For ELISA with pre-coated antigens, the ELISA plates were pre-coated with 100 μL per well of WT115-hPro1.ECD.His (2 μg/mL) or truncated D3 proteins (2 μg/mL or 5 μg/mL) at 4° C. overnight. The antigen was diluted in coating buffer (0.02 M Na2CO3 and 0.18 M NaHCO₃, pH9.2) from stock solution. Next day, the plates were washed using 1×PBST for one time, and blocking was done by using 200 μL of 1×PBS/2% BSA. Constant concentration of antibody (2 μg/mL) was diluted in blocking buffer. After 1-hour blocking, the plates were washed using 1×PBST for 3 times, and then antibody was added to the plates and incubated at ambient temperature for 1 hour. Binding of antibodies to the immobilized human D3 was detected by HRP-labeled secondary antibody (Bethyl, A80-304P), which was diluted in 1×PBS/2% BSA at a concentration of 1:10000. After incubation, the plates were washed using 1×PBST for 6 times. The color was developed by dispensing 100 μL of TMB substrate, and then reaction stopped by adding 100 μL of 2M HCl. Absorbance was read at 450 nm and 540 nm using a microplate spectrophotometer. All samples were tested in duplicate. The results are shown in FIG. 7b.

As shown in FIGS. 7a and 7b, WT1156-P3R2-1C2-uIgG1 binds to WT115-hPro1.V1.ECD.MBP.AVI.His, WT115-hPro.V2.ECD.MBP.AVI.His and partially to WT115-hPro1.V3.ECD.MBP.AVI.His, but not to the other isoforms, indicating its binding epitope is located in EGF1-2. WT1156-P3R2-1C9-uIgG1 and WT1156-P3R2-1H6-uIgG1 bind to WT115-hPro1.ECD.His, but not to truncated D3 isoforms, indicating the binding epitope of these two antibodies is located in N-term. For WT1156-P8R2-1H1-uIgG1, when tested by ELISA with pre-coated antibodies, it shows binding to WT115-hPro1.ECD.His, but not to truncated D3 isoforms (FIG. 7a); when tested by ELISA with pre-coated antigens, as shown in FIG. 7b, WT1156-P8R2-1H1-uIgG1 shows binding to WT115-hPro1.V1.ECD.MBP.AVI.His and WT115-hPro1.V2.ECD.MBP.AVI.His, but not to the other isoforms, indicating its binding epitope is probably located in N-term-DSL-EGF-1. The ELISA binding results show that WT115-BMK1 binds to DSL domain and WT115-BMK2 binds to EGF-3 domain, which are consistent with the results shown in US 2019/0046656 and WO 2017/021349, respectively.

### 3.9 Cross-Family Binding of Anti-Human D3 Antibodies

Plates were pre-coated with 1 μg/mL, 100 μL per well of WT115-hPro1.ECD.His, WT115-hPro2.ECD.His (human D1, SinoBiological, Cat: 11635-H08H) or WT115-hPro3.ECD.His (human D4, SinoBiological, Cat: 10171-H08H) at 4° C. overnight. The antigen was diluted in coating buffer (0.02 M Na2CO3 and 0.18 M NaHCO₃, pH9.2) from stock solution. Next day, the plates were washed using 1×PBST (PBS containing 0.05% tween-20) for one time, and blocking was done by adding 200 μL of 1×PBS/2% BSA per well. During blocking, BMKs and WT1156 antibodies were diluted to 10 nM in blocking buffer, and WT115-cAbs (WT115-cAb1 is anti-D1 antibody purchased from SinoBiological, Cat: 11635-MM07; WT115-cAb2 is anti-D4 antibody purchased from SinoBiological, Cat: 10171-MM15) were diluted 1000 fold. After 1-hour blocking, the plates were washed using 1×PBST for 3 times, and then the diluted antibodies were added to the plates and incubated at ambient temperature for 1 hour. Binding of antibodies to the immobilized human D3 was detected by Goat anti-human IgG-Fc Fragment Cross-absorbed Antibody HRP (Bethyl, A80-304P) and Mouse IgG-Fc Fragment cross-adsorbed Antibody HRP (Bethyl, A90-231P), which were diluted in 1×PBS/2% BSA at 1:10000. After incubation, the plates were washed using 1×PBST for 6 times. The color was developed by dispensing 100 μL of TMB substrate, and then reaction was stopped by adding 100 μL of 2M HCl. Absorbance was read at 450 nm and 540 nm using a microplate spectrophotometer. WT115-cAb1 and cAb2 were used as anti-human D1 and D4 positive control respectively. Human IgG1 isotype antibody was used as a negative control. All samples were tested in duplicate.

As shown in FIG. 8, WBPT1156 antibodies do not bind to human D1 or human D4.

### 3.10 Human Serum Stability

Human serum was freshly isolated from healthy donors by centrifugation. The samples were diluted in serum and serum volume accounts for more than 90% of total volume. Five aliquots of the sample were incubated at 37° C. Samples were then collected at day 0, day 1, day 4, day 7 and day 14, respectively and quick-frozen until analysis together.

The stability of the samples was tested by binding to human D3 using ELISA. Briefly, plates were pre-coated with 100 μL/well of 1 μg/mL WT115-hPro1.ECD.His at 4° C. overnight. Next day, the plates were washed using 1×PBST (PBS containing 0.05% tween-20) for one time, and blocking was done by adding 200 μL of 1×PBS/2% BSA per well. During blocking, testing antibodies were added to the plates at various concentrations (4-fold serially diluted from 3 nM to 0.00018 nM). The plates were incubated at ambient temperature for 1 hour. Binding of antibodies to the immobilized human D3 was detected by Goat anti-human IgG-Fc Fragment Cross-absorbed Antibody HRP (Bethyl, A80-304P) and Mouse IgG-Fc Fragment cross-adsorbed Antibody HRP (Bethyl, A90-231P), which were diluted in 1×PBS/2% BSA at 1:5000. After incubation, the plates were washed using 1×PBST for 6 times. The color was developed by dispensing 100 μL of TMB substrate, and then reaction was stopped by adding 100 μL of 2M HCl. Absorbance was read at 450 nm and 540 nm using a microplate spectrophotometer. Human IgG1 isotype antibody was used as a negative control. All samples were tested in duplicate.

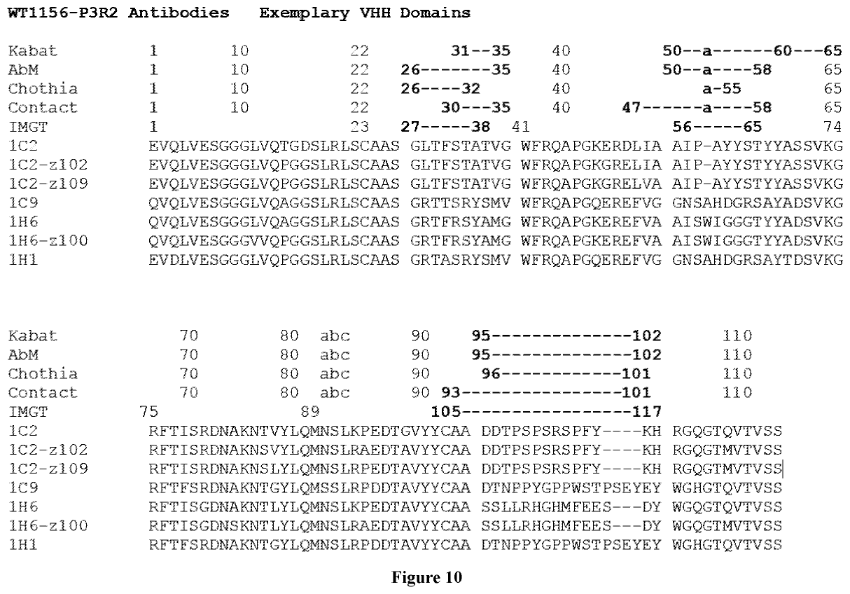

As shown in FIG. 9, after incubation in human serum at 37° C. for up to 14 days, the binding profile of WT1156-P3R2-1C2-z109-uIgG1 to human D3 protein did not change.

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

1. A D3-binding molecule comprising an immunoglobulin single variable domain, wherein the single variable domain comprises a CDR1, CDR2, and CDR3 of a VHH as set forth in SEQ ID NO: 12, 13, 14, 15, 16, 17, 55 or 18.

2. The D3-binding molecule of claim 1, wherein CDR1, CDR2, and CDR3 are according to Kabat, Chothia, AbM, Contact, IMGT, or any combination(s) thereof.

3. The D3-binding molecule of claim 1, wherein (i) the CDR1 comprises an amino acid sequence as set forth in SEQ ID NO: 1, 4, 7 or 10; (ii) the CDR2 comprises an amino acid sequence as set forth in SEQ ID NO: 2, 5, 8 or 11; and (iii) the CDR3 comprises an amino acid sequence as set forth in SEQ ID NO: 3, 6 or 9.

4. The D3-binding molecule of claim 1, wherein the D3-binding molecule comprises:
(A) a CDR1 as set forth in SEQ ID NO: 1; a CDR2 as set forth in SEQ ID NO: 2; and a CDR3 as set forth in SEQ ID NO: 3;
(B) a CDR1 as set forth in SEQ ID NO: 4; a CDR2 as set forth in SEQ ID NO: 5; and a CDR3 as set forth in SEQ ID NO: 6;
(C) a CDR1 as set forth in SEQ ID NO: 7; a CDR2 as set forth in SEQ ID NO: 8; and a CDR3 as set forth in SEQ ID NO: 9; or
(D) a CDR1 as set forth in SEQ ID NO: 10; a CDR2 as set forth in SEQ ID NO: 11; and a CDR3 as set forth in SEQ ID NO: 6.

5. The D3-binding molecule of any of claims 1 to 4, wherein the single variable domain comprises:
(A) the amino acid sequence as set forth in any one of SEQ ID NOs: 12-18 and 55;
(B) an amino acid sequence at least 85%, 90%, or 95% identical to the amino acid sequence as set forth in any one of SEQ ID NOs: 12-18 and 55 yet retaining the specific binding affinity to D3; or
(C) an amino acid sequence with addition, deletion and/or substitution of one or more (e.g. 1, 2 or 3) amino acids compared with the amino acid sequence as set forth in any one of SEQ ID NOs: 12-18 and 55.

6. The D3-binding molecule of any of claims 1 to 5, comprising one or more substitutions, additions and/or deletions of amino acids in the framework regions, e.g. FRW1, FRW2, FRW3, and/or FRW4 of the single variable domain.

7. The D3-binding molecule of any of claims 1 to 6, wherein the single variable domain comprises the amino acid sequence as set forth in any one of SEQ ID NOs: 12-18 and 55.

8. The D3-binding molecule of any of claims 1 to 7, wherein the D3-binding molecule further comprises a human IgG constant domain.

9. The D3-binding molecule of claim 8, wherein the human IgG constant domain is a human IgG1, IgG2, IgG3 or IgG4 constant domain, such as a human IgG1 constant domain or a variant thereof.

10. The D3-binding molecule of any of claims 1 to 9, which has one or more of the following properties:
(a) bind to human D3, cyno D3 and/or mouse D3 with EC50s at nM grade, as measured by ELISA or FACS;
(b) show dose-dependent internalization potency in human D3 expressing cells; and
(c) bind to human D3 with a KD of no more than 0.1 nM, as measured by SPR.

11. The D3-binding molecule of any of claims 1 to 10, wherein the D3-binding molecule is a chimeric antibody, a humanized antibody or a fully human antibody.

12. The D3-binding molecule of any of claims 1 to 11, which comprises a single variable domain as set forth in any one of SEQ ID NOs: 12-18 and 55, and an IgG constant domain as set forth in SEQ ID NO: 19.

13. The D3-binding molecule of any of claims 1 to 12, which is a dimer.

14. A fusion protein comprising the D3-binding molecule as defined in any of claims 1 to 13 fused to a heterologous peptide, such as an antigen-binding domain targeting a different antigen.

15. A nucleic acid molecule comprising a nucleic acid sequence encoding the single variable domain of the D3-binding molecule as defined in any of claims 1 to 13.

16. A vector comprising the nucleic acid molecule of claim 15.

17. A host cell comprising the vector of claim 16.

18. A pharmaceutical composition comprising at least one D3-binding molecule as defined in any of claims 1 to 13 and a pharmaceutically acceptable carrier.

19. A method for producing the D3-binding molecule as defined in any of claims 1 to 13 comprising the steps of:
expressing the D3-binding molecule in the host cell of claim 15; and
isolating the D3-binding molecule from the host cell.

20. A method of modulating a D3 related immune response in a subject, comprising administering to the subject the D3-binding molecule as defined in any of claims 1 to 13 or the pharmaceutical composition of claim 18 such that an immune response is modulated in the subject.

21. A method for treating or preventing cancer in a subject, comprising administering an effective amount of the D3-binding molecule as defined in any of claims 1 to 13 or the pharmaceutical composition of claim 18 to the subject, wherein the cancer is D3 positive or overexpressed.

22. The method of claim 21, wherein the cancer is selected from lung cancer and neuroendocrine carcinoma.

23. The method of claim 22, wherein the cancer is SCLC or LCNEC.

24. Use of the D3-binding molecule as defined in any of claims 1 to 13 in the manufacture of a medicament for diagnosing, preventing or treating D3 positive cancer.

25. D3-binding molecule as defined in any of claims 1 to 13 for use in treating or preventing D3 positive cancer.

26. A kit for treating or diagnosing cancer, comprising a container comprising the D3-binding molecule as defined in any of claims 1 to 13.


SEQUENCE LISTING (56 sequences)

SEQ ID NO: 1 - protein, 10 aa, synthetic construct
  REGION 1..10: note = CDR1
GLTFSTATVG

SEQ ID NO: 2 - protein, 16 aa, synthetic construct
  REGION 1..16: note = CDR2
AIPAYYSTYYASSVKG

SEQ ID NO: 3 - protein, 14 aa, synthetic construct
  REGION 1..14: note = CDR3
DDTPSPSRSPFYKH

SEQ ID NO: 4 - protein, 10 aa, synthetic construct
  REGION 1..10: note = CDR1
GRTTSRYSMV

SEQ ID NO: 5 - protein, 17 aa, synthetic construct
  REGION 1..17: note = CDR2
GNSAHDGRSAYADSVKG

SEQ ID NO: 6 - protein, 18 aa, synthetic construct
  REGION 1..18: note = CDR3
DTNPPYGPPWSTPSEYEY

SEQ ID NO: 7 - protein, 10 aa, synthetic construct
  REGION 1..10: note = CDR1
GRTFRSYAMG

SEQ ID NO: 8 - protein, 17 aa, synthetic construct
  REGION 1..17: note = CDR2
AISWIGGGTYYADSVKG

SEQ ID NO: 9 - protein, 15 aa, synthetic construct
  REGION 1..15: note = CDR3
SSLLRHGHMFEESDY

SEQ ID NO: 10 - protein, 10 aa, synthetic construct
  REGION 1..10: note = CDR1
GRTASRYSMV

SEQ ID NO: 11 - protein, 17 aa, synthetic construct
  REGION 1..17: note = CDR2
GNSAHDGRSAYTDSVKG

SEQ ID NO: 12 - protein, 122 aa, synthetic construct
  REGION 1..122: note = VHH
EVQLVESGGGLVQTGDSLRLSCAASGLTFSTATVGWFRQAPGKERDLIAAIPAYYSTYYASSVKGRFTISRDNAKNTVYLQMNSLKPEDTGVYYCAADDTPSPSRSPFYKHRGQGTQVTVSS

SEQ ID NO: 13 - protein, 127 aa, synthetic construct
  REGION 1..127: note = VHH
QVQLVESGGGLVQAGGSLRLSCAASGRTTSRYSMVWFRQAPGQEREFVGGNSAHDGRSAYADSVKGRFTFSRDNAKNTGYLQMSSLRPDDTAVYYCAADTNPPYGPPWSTPSEYEYWGHGTQVTVSS

SEQ ID NO: 14 - protein, 124 aa, synthetic construct
  REGION 1..124: note = VHH
QVQLVESGGGLVQAGGSLRLSCAASGRTFRSYAMGWFRQAPGKEREFVAAISWIGGGTYYADSVKGRFTISGDNAKNTLYLQMNSLKPEDTAVYYCAASSLLRHGHMFEESDYWGQGTQVTVSS

SEQ ID NO: 15 - protein, 127 aa, synthetic construct
  REGION 1..127: note = VHH
EVDLVESGGGLVQPGGSLRLSCAASGRTASRYSMVWFRQAPGQEREFVGGNSAHDGRSAYTDSVKGRFTFSRDNAKNTGYLQMNSLRPDDTAVYYCAADTNPPYGPPWSTPSEYEYWGHGTQVTVSS

SEQ ID NO: 16 - protein, 122 aa, synthetic construct
  REGION 1..122: note = VHH
EVQLVESGGGLVQPGGSLRLSCAASGLTFSTATVGWFRQAPGKGRELIAAIPAYYSTYYASSVKGRFTISRDNAKNSVYLQMNSLRAEDTAVYYCAADDTPSPSRSPFYKHRGQGTMVTVSS

SEQ ID NO: 17 - protein, 122 aa, synthetic construct
  REGION 1..122: note = VHH
EVQLVESGGGLVQPGGSLRLSCAASGLTFSTATVGWFRQAPGKGRELVAAIPAYYSTYYASSVKGRFTISRDNAKNSLYLQMNSLRPEDTAVYYCAADDTPSPSRSPFYKHRGQGTMVTVSS

SEQ ID NO: 18 - protein, 124 aa, synthetic construct
  REGION 1..124: note = VHH
QVQLVESGGGVVQPGGSLRLSCAASGRTFRSYAMGWFRQAPGKEREFVAAISWIGGGTYYADSVKGRFTISGDNSKNTLYLQMNSLRAEDTAVYYCAASSLLRHGHMFEESDYWGQGTMVTVSS

SEQ ID NO: 19 - protein, 227 aa, synthetic construct
  REGION 1..227: note = IgG1 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 8 aa, synthetic construct
  REGION 1..8: note = CDR1
GLTFSTAT

SEQ ID NO: 21 - protein, 7 aa, synthetic construct
  REGION 1..7: note = CDR2
IPAYYST

SEQ ID NO: 22 - protein, 16 aa, synthetic construct
  REGION 1..16: note = CDR3
AADDTPSPSRSPFYKH

SEQ ID NO: 23 - protein, 5 aa, synthetic construct
  REGION 1..5: note = CDR1
TATVG

SEQ ID NO: 24 - protein, 7 aa, synthetic construct
  REGION 1..7: note = CDR1
GLTFSTA

SEQ ID NO: 25 - skipped

SEQ ID NO: 26 - protein, 12 aa, synthetic construct
  REGION 1..12: note = CDR3
DTPSPSRSPFYK

SEQ ID NO: 27 - protein, 6 aa, synthetic construct
  REGION 1..6: note = CDR1
STATVG

SEQ ID NO: 28 - protein, 12 aa, synthetic construct
  REGION 1..12: note = CDR2
LIAAIPAYYSTY

SEQ ID NO: 29 - protein, 15 aa, synthetic construct
  REGION 1..15: note = CDR3
AADDTPSPSRSPFYK

SEQ ID NO: 30 - protein, 9 aa, synthetic construct
  REGION 1..9: note = CDR2
AIPAYYSTY

SEQ ID NO: 31 - protein, 8 aa, synthetic construct
  REGION 1..8: note = CDR1
GRTTSRYS

SEQ ID NO: 32 - protein, 8 aa, synthetic construct
  REGION 1..8: note = CDR2
NSAHDGRS

SEQ ID NO: 33 - protein, 20 aa, synthetic construct
  REGION 1..20: note = CDR3
AADTNPPYGPPWSTPSEYEY

SEQ ID NO: 34 - protein, 5 aa, synthetic construct
  REGION 1..5: note = CDR1
RYSMV

SEQ ID NO: 35 - protein, 7 aa, synthetic construct
  REGION 1..7: note = CDR1
GRTTSRY

SEQ ID NO: 36 - protein, 4 aa, synthetic construct
  REGION 1..4: note = CDR2
AHDG

SEQ ID NO: 37 - protein, 16 aa, synthetic construct
  REGION 1..16: note = CDR3
TNPPYGPPWSTPSEYE

SEQ ID NO: 38 - protein, 6 aa, synthetic construct
  REGION 1..6: note = CDR1
SRYSMV

SEQ ID NO: 39 - protein, 13 aa, synthetic construct
  REGION 1..13: note = CDR2
FVGGNSAHDGRSA

SEQ ID NO: 40 - protein, 19 aa, synthetic construct
  REGION 1..19: note = CDR3
AADTNPPYGPPWSTPSEYE

SEQ ID NO: 41 - protein, 10 aa, synthetic construct
  REGION 1..10: note = CDR2
GNSAHDGRSA

SEQ ID NO: 42 - protein, 8 aa, synthetic construct
  REGION 1..8: note = CDR1
GRTFRSYA

SEQ ID NO: 43 - protein, 8 aa, synthetic construct
  REGION 1..8: note = CDR2
ISWIGGGT

SEQ ID NO: 44 - protein, 17 aa, synthetic construct
  REGION 1..17: note = CDR3
AASSLLRHGHMFEESDY

SEQ ID NO: 45 - protein, 5 aa, synthetic construct
  REGION 1..5: note = CDR1
SYAMG

SEQ ID NO: 46 - protein, 7 aa, synthetic construct
  REGION 1..7: note = CDR1
GRTFRSY

SEQ ID NO: 47 - protein, 4 aa, synthetic construct
  REGION 1..4: note = CDR2
WIGG

SEQ ID NO: 48 - protein, 13 aa, synthetic construct
  REGION 1..13: note = CDR3
SLLRHGHMFEESD

SEQ ID NO: 49 - protein, 6 aa, synthetic construct
  REGION 1..6: note = CDR1
RSYAMG

SEQ ID NO: 50 - protein, 13 aa, synthetic construct
  REGION 1..13: note = CDR2
FVAAISWIGGGTY

SEQ ID NO: 51 - protein, 16 aa, synthetic construct
  REGION 1..16: note = CDR3
AASSLLRHGHMFEESD

SEQ ID NO: 52 - protein, 10 aa, synthetic construct
  REGION 1..10: note = CDR2
AISWIGGGTY

SEQ ID NO: 53 - protein, 8 aa, synthetic construct
  REGION 1..8: note = CDR1
GRTASRYS

SEQ ID NO: 54 - protein, 7 aa, synthetic construct
  REGION 1..7: note = CDR1
GRTASRY

SEQ ID NO: 55 - protein, 122 aa, synthetic construct
  REGION 1..122: note = VHH
EVQLVESGGGLVQPGGSLRLSCAASGLTFSTATVGWFRQAPGKGRELIAAIPAYYSTYYASSVKGRFTISRDNAKNSLYLQMNSLRPEDTAVYYCAADDTPSPSRSPFYKHRGQGTMVTVSS

SEQ ID NO: 56 - protein, 12 aa, synthetic construct
LVAAIPAYYSTY